(19) United States
(12) Patent Application Publication
(10) Pub. No.: US 2018/0289821 A1
(43) Pub. Date: Oct. 11, 2018

(54) Oxazoline Polymer Compositions and Use Thereof

(71) Applicants: Ray Yin, Wilmington, DE (US); Jing Pan, Newark, DE (US); Dujie Qin, Wilmington, DE (US); Yubei Zhang, Hockessin, DE (US)

(72) Inventors: Ray Yin, Wilmington, DE (US); Jing Pan, Newark, DE (US); Dujie Qin, Wilmington, DE (US); Yubei Zhang, Hockessin, DE (US)

(73) Assignee: ANP Technologies, Inc., Newark, DE (US)

(21) Appl. No.: 16/010,419

(22) Filed: Jun. 16, 2018

Related U.S. Application Data
(63) Continuation-in-part of application No. 14/236,758, filed on Oct. 2, 2014.
(60) Provisional application No. 61/514,880, filed on Aug. 3, 2011.

(51) Int. Cl. A61K 47/60; A61K 47/68; A61K 47/65; A61K 47/12

(52) U.S. Cl. CPC A61K 47/60; A61K 47/6809; A61K 47/12; A61K 47/6883; A61K 47/65; A61K 47/6851

(57) ABSTRACT

Compositions comprising an oxazoline polymer and optional linkers to carry a variety of molecules.

### CROSS REFERENCE

The is a continuation-in-part of U.S. application Ser. No.: 14/236,758 filed on Oct. 2, 2014, now pending, which is a U.S. national application of PCT/US2012/049410 filed on Aug. 3, 2012, which claims the priority of U.S. Provisional Application Ser. No.: 61/514,880, filed on Aug. 3, 2011, and which is hereby incorporated by reference.

### FIELD OF THE INVENTION

The present invention concerns the use of an end-functionalized polyoxazoline polymer comprising, for example, a poly (unsubstituted oxazoline), a poly (substituted oxazoline) polymer or a combination thereof, wherein said polymer can be a linear, branched, randomly branched or dendritically branched polymer. The reactive chain ends of the polyoxazoline polymer, for example, poly (2-methyloxazoline) (PMOX), poly (2-ethyoxazoline) (PEOX), poly (2-propyloxazoline) (PPDX) and poly (2-butyloxazoline) (PBOX), can comprise a small molecule comprising multifunctional, unprotected amino groups or imino groups. Such an end-functionalized polymer then can be linked to or associated with a bioactive material through an additional linker molecule either directly or indirectly, or by physically mixing the polymer and bioactive material bringing the two into association. Alternatively, the initiator portion of said polymer also can be linked with said bioactive material. The resulting composition can be employed in agriculture, environmental studies, diagnostics, drug monitoring, drug target screening, drug lead optimization and therapeutics, for example.

### BACKGROUND

### Polyoxazoline Polymers

Polyoxazoline (PDX) polymers have been used in cosmetic and food packaging applications. Due to good water solubility, PDX also has been considered a candidate to replace polyethyleneglycol (PEG) for different biomedical-related applications (Adams, Advanced Drug Delivery Reviews 59 (2007) 1504-1520 and Mero, Journal of Controlled Release 125 (2008) 87-95).

POX polymers comprising, for example, poly (unsubstituted oxazoline) or poly (substituted oxazoline), can be produced by cationic ring opening polymerization. Commonly used water-soluble polymers are PMOX and PEOX. Under living polymerization conditions (e.g., conditions including fast initiation, slow propagation and the lack of chain termination and transfer reactions), a well defined, linear PMOX and PEOX can be produced (Tomalia et al., Macromol. 1991, 24, 1435. Kobayashi, J. Polym. Sci. Part A: Polym. Chem.: Vol. 40 (2002)); while under other synthesis conditions, branched or randomly branched PMOX and PEOX polymers can be generated (Litt, Macromol. Sci. Chem. A9(5), 703-727 (1975) and Yin, U.S. Pat. No. 7,754,500).

Under both types of reaction conditions, an electrophilic (e.g., cationic) chain end can be generated and further reacted with a nucleophilic group or a molecule containing a nucleophilic group so that the polymerization reaction can be terminated. Most known methods for terminating such a reactive chain end use a monofunctional nucleophilic group, such as those consisting of a single imino (——NH——) group, for example, a morpholine or a protected piperazine (Tomalia, U.S. Pat. No. 5,773,527 and Zhang et. al., Macromol., 2009, 42 (6) 2215-2221). That is true for the termination of linear POX polymers, such as a living linear PMOX or PEOX, where a defined chain end can be generated.

The termination of a reactive PDX polymer with a polyfunctional polymer to generate star, comb, Starburst or Combburst polymers is described, for example, in Tomalia, U.S. Pat. No. 5,773,527.

However, use of a multifunctional small molecule without any protecting groups to terminate a reactive POX to generate a functional polymer with only one polymer per terminating molecule is not a preferable or a desired way to make a functionalized POX. That approach tends to produce more dimeric and multimeric POX blocks, such as, star-branched and comb-branched polymers.

### Symmetrically Branched (SB) Polymers (SBP) and Asymmetrically Branched (AB) Polymers (ABP)

In recent years, dendritic polymers, including Starburst dendrimers (or Dense Star polymers) and Combburst dendrigrafts (or hyper comb-branched polymers), have been developed for a variety of applications (“Dendritic Molecules” ed. by Newkome et al., VCH, Weinheim, 1996 and “Dendrimers and Other Dendritic Polymers” ed. by Frechet & Tomalia, John Wiley & Sons, Ltd., 2001). Those polymers exhibit: (a) a well-defined core molecule, (b) at least two concentric dendritic layers (generations) with symmetrical (equal) branch junctures and (c) exterior surface groups, as described in U.S. Pat. Nos. 4,435,548; 4,507,466; 4,568,737; 4,587,329; 5,338,532; 5,527,524; and 5,714,166, and the references cited therein.

SB dendrimers also are distinctively different from the previously prepared AB dendrimers (U.S. Pat. Nos. 4,289,872; 4,360,646; and 4,410,688 of Denkewalter) which possess asymmetrical (unequal) branch junctures.

Both types of dendrimers can be produced by repetitive protecting and deprotecting procedures through either a divergent or a convergent synthetic approach. Since SB and AB dendrimers utilize small molecules as building blocks for the core and the branches, the molecular weights of such dendrimers often are precisely defined. In the case of lower generation molecules, a single molecular weight dendrimer often is obtained.

Similar to dendrimers, Combburst dendrigrafts also are constructed with a core and concentric layers with symmetrical branches through a stepwise synthetic method. In contrast to dendrimers, Combburst dendrigrafts or polymers are generated with monodisperse linear polymeric building blocks (Tomalia, U.S. Pat. No. 5,773,527 and Yin, U.S. Pat. Nos. 5,631,329 and 5,919,442). Moreover, the branch pattern also is different from that of dendrimers. For example, Combburst dendrigrafts form branch junctures along the polymeric backbones (chain branches) while Starburst dendrimers often branch at the termini (terminal branches). Due to the utilization of living polymerization techniques, the molecular weight distribution (M_{w}/Mₙ) of such polymeric building blocks (core and branches) often is narrow. As a result, Combburst dendrigrafts, produced through a graft-on-graft process, are rather well defined with an M_{w}/Mₙ often less than 1.2.

Although possessing well controlled molecular architecture, such as, well defined size, shape and surface functional groups, both dendrimers and dendrigrafts can be produced only through a large number of reiteration steps, making such useful only for academic pursuits rather than large scale commercial applications.

Dendrimers and dendrigrafts can serve as carriers for bioactive molecules, as described in U.S. Pat. Nos. 5,338,532; 5,527,524; and 5,714,166 of Tomalia for dense star polymers and U.S. Pat. No. 5,919,442 of Yin for hyper comb-branched polymers. The surface functional groups and interior void spaces of those molecules have been suggested as a basis for the carrier property, for example, due to the well-controlled, symmetrical dendritic architecture with predictable branching patterns (either symmetrical termini or polymeric chain branching) and molecular weight.

The preparation of regular (reg) asymmetrically branched polymer (reg-ABP) made of polylysine has been described, as illustrated in U.S. Pat. Nos. 4,289,872; 4,360,646; and 4,410,688.

The synthesis and mechanisms of random (ran) asymmetrically branched polymers (ran-ABP), such as, made of polyethyleneimine (PEI), have been described (see Jones et al., J. Org. Chem. 9, 125 (1944), Jones et al., J. Org. Chem. 30, 1994 (1965) and Dick et al., J. Macromol. Sci. Chem., A4 (6), 1301-1314, (1970)).

The synthesis and characterization of ran-ABP, such as made of POX, for example, PMOX or PEOX, have been described by Litt (J. Macromol. Sci. Chem. A9(5), pp. 703-727 (1975)) and Warakomski (J. Polym. Sci. Polym. Chem. 28, 3551 (1990)).

Randomly branched PEOX has been utilized to physically encapsulate protein molecules (U.S. Pat. No. 6,716,450). However, such an approach was not designed for the direct, covalent linking of ABP with bioactive materials for bioassays and drug delivery applications.

### Polymer-Bioactive Material (BM) Compositions

Polymer-bioactive material (BM) compositions, such as, PEG or polyethyleneoxide (PEO)-drug compositions, including directly or indirectly linked conjugates, or physical mixtures of PEG/PEO and drug are known. Although less extensively studied, POX-drug compositions also have been reported, including a linear polymer drug composition, such as those described in Mero et al. in J. Contr. Rel. 125 (2008) 87-95 and Viegas et al., Bioconj. Chem. 2011, 22, 976-986, as well as dendritic polymer drug compositions, such as those described by Yin in U.S. Pat. No. 5,919,442.

Special protective chemistries were used during the termination step (Hsiue, Bioconj. Chem. 2006, 17, 781-786, U.S. Pat. No. 7,943,141, U.S. Pub. No. 2011/0123453 and Zhang, et al., Macromol. 2009, 42 (6) 2215-2221). However, none of those approaches utilized an unprotected, multifunctional small terminating molecule for the in situ functionalization of linear POX polymers, which can significantly reduce production costs.

### Assays and Microarrays

Since completion of the human genome project, it has become evident that elucidation of biological pathways and mechanisms at the protein level can be as important as studies at the genetic level because the former is more closely associated with disease and disease states, as well as the treatment thereof. With that strong demand, a new forum called proteomics developed and that art is a major focus of industrial and academic pursuits.

Currently, three major research areas of proteomics studies include drug discovery, high throughput screening and validation of new protein targets and drug leads. Tools include two dimensional (2-D) gel electrophoresis, mass spectrometry, and more recently, protein microarrays. In contrast to the lengthy 2-D gel procedures and tedious sample preparation (primarily separations) involved in mass spectrometry analysis, protein microarrays provide a quick, generally simple and low cost method to screen large amounts of proteins and the functions thereof. Therefore, microarrays are developing as desirable tools in proteomics.

However, protein-based microarray technology is far less developed than is gene microarray technology. The construction of a protein/antibody chip presents daunting challenges not encountered in the development of classical immunoassays or of DNA chips. For example, proteins are more sensitive to the environment than are nucleic acids. The hydrophobicity of many membrane, glass and plastic surfaces can cause protein denaturation destroying the structure and/or function thereof thereby rendering a protein reagent structurally and/or functionally inactive, which can result in lower assay sensitivity and higher signal-to-noise ratios. In other words, to construct a protein microarray, at least three issues must be addressed, protein denaturation, protein immobilization and protein orientation.

For example, a protein molecule often folds into a three-dimensional (3-D) structure in solution for and to maintain biological activity. On interaction with different solid surfaces, for example, during immobilization of proteins onto membranes, glass slides or micro/nanoparticles, the 3-D structure of the protein molecule often collapses thereby often destroying biological activity or at least functional structures. In addition, proteins often do not have the ability to adhere onto different surfaces.

To immobilize a protein on a surface, direct covalent linking reactions or electrostatic interactions (physical adsorption) often are employed. But, heterogeneous chemical reactions often are incomplete yielding undesired side products (i.e. incomplete modification of surfaces) and in some cases, also partially denatured proteins during different reaction stages.

Electrostatic interaction relies heavily on the isoelectric points of the proteins, as well as the pH of the buffer solutions.

Both approaches tend to yield irreproducible results due to the complexity of those procedures. Lot-to-lot reproducibility is, therefore, very poor.

As a result, there is interest in modifying solid substrates, but not the protein molecule, to obtain surfaces carrying biologically active protein. A variety of polymers, including PEI polymers, have been utilized as coating materials to alter the characteristics of solid surfaces for the construction of protein arrays, Wagner et al., U.S. Pat. No. 6,406,921.

### SUMMARY

The present invention is directed to a functionalized polymer comprising a polyoxazoline polymer P reacted with at least one initiator I and a first linker L, the L is further reacted with two or more second linker M, wherein, the initiator I comprises a first functional group that is reacted with P and a second functional group that is reactive with a first biologically active molecule B1; the first linker L comprises three or more active hydrogens in amine groups selected from an NH₃, two or more —NH₂ groups, three or more imino (—NH—) groups, or a combination of —NH₂ and —NH— groups, wherein the first linker L is reacted with the P with one of the active hydrogens; the M comprises a carbodiimide (CDI), a CDI-functionalized molecule, glycidol (G), succinic anhydride (SA), acrylic ester, acrylamide or a heterofunctional molecule, the M comprises at least one first linker group reacted with one of the active hydrogens of the L and at least one second linker group reactive with a second biologically active molecule B2.

In one aspect, the present invention is directed to terminating a reactive end of a polyoxazoline (POX) polymer with an excess of a multi-functional small molecule with an unprotected amino or imino group so that a one-to-one adduct (one polymer per terminating molecule) can be formed. For example, a POX polymer can comprise the following configuration: Initiator (Iₙ)-POX (P)-End Group (Eₘ), where m and n each is ≥1. On purification, for example, solvent precipitation and/or dialysis, the purified amino-end functionalized (—NH₂) or imino-end functionalized (—NH—) POX can be further linked to a pharmaceutically active agent (PAA) through additional linkers to form a covalently linked composition for diagnostic and/or therapeutic uses.

Suitable linker molecules include carbodiimidazole (CDI) or a partial CDI-functionalized molecule (e.g., a reaction product between a CDI and an OH-functionalized molecule or a bioactive material (BM)), glycidol, succinic anhydride, acrylic ester, amidoamine, linear or branched polyamidoamine, acrylamide or an N-hydroxysuccinimide (NHS)-containing heterofunctional molecule to produce a POX-BM composition or a POX polymer with different functional groups, such as, —OH, —COON, —COONa, ester, amide, maleimide or —SH that can be linked with a BM. In other words, a BM can be linked to the functionalized POX polymers either directly or indirectly through the functional groups.

In another aspect, in embodiments, a biological material, a bioactive material, a pharmaceutically active agent and the like can be complexed with the polymer of interest without a formal reaction resulting in a covalent bond, instead mere mixing of the polymer with the a biological material, a bioactive material, a pharmaceutically active agent and the like results in a physical relationship between same and a polymer of interest such that the two entities demonstrate a coordinated presence. Hence, such a composition without a covalent bond of a biological material, a bioactive material, a pharmaceutically active agent and the like and a polymer of interest has the same properties and functions as other compositions of interest.

In another aspect of the invention, the initiator moiety (I) incorporated into POX polymers may include various functional/protected functional groups. On reaction/deprotection, a second functional group at the initiator end, particularly with a different functionality as that of the end group previously functionalized, can be formed at the initiator end, which can be used for the attachment of an additional BM on the same polymer. For example, in addition to the BM already attached to a POX at the reactive chain end, an additional BM also can be attached to the same polymer at the initiator end, if the POX utilized is linear. If the POX is branched, multiple BM molecules can be attached to the same polymer at the multiple initiator ends. In addition, due to the stepwise reaction approach, not only the same but different BM molecules can be attached to the same POX polymer at the initiator and reactive chain ends. Such a differentiated POX can be useful in linking different kinds of BM molecules, and at varying ratios.

Accordingly, various other functional groups can be introduced at the initiator end of the POX polymer in addition to at the terminator end. The functional groups include, but are not limited to, ethyl bromoacetate, methyl bromoacetate, bromoacetone, tert-butyl bromoacetate, propyl bromoacetate, benzyl bromoacetate, sulfur-containing compounds, such as, 2-(p-toluenesulfonyloxy) ethyl disulfide (TOEDS), (chloromethyl) methyl disulfide, bis(iodomethyl)methyl disulfide and 2-bromoethyl disulfide, silicon-containing compounds, such as, (3-chloropropyl)triethoxysilane, (3-bromopropyl) trimethoxysilane and (3-iodopropyl)trimethoxysilane and protected groups for amines or imines, such as, those comprising a carboxybenzyl group, a p-methoxybenzyl carbonyl group, a tert-butyloxycarbonyl group and a 9-fluorenylmethyloxycarbonyl (FMOC) group; and so on.

For example, TOEDS, a difunctional initiator, can be utilized to initiate the polymerization of oxazoline monomers at both ends. On termination, for example, with a large excess of ethylene diamine (EDA), a POX polymer with amino and imino functional groups at both chain ends can be produced. The chain ends further can be linked with any of a plurality of bioactive materials, such as, small molecule drugs, such as, gemcitabine, camptothecin, paclitaxel and so on, either directly or indirectly through covalent linkages. When the reagents comprise a disulfide bond, addition of a reducing agent, such as, dithiothreitol (DTT), can cleave that bond to generate a sulfhydryl-functionalized POX polymer-bioactive material composition. The sulfhydryl group then can be linked, for example, with a maleimide-functionalized targeting molecule, such as, peptide, protein, such as, antibody, sialic acid, one member of a binding pair and so on to provide a differentiated POX polymer composition with one end linked with any of a plurality of bioactive materials, such as, small molecule drugs, and the other end linked with at least a targeting molecule, such as, peptide, protein, such as, antibody, sialic acid and so on. Various differentiated POX compositions, including, but not limited to, biologically active molecules to generate, such as, BAM-POX-PAA, BAM₁,-POX-BAM₂, PAA,POX-PAA₂ and so on are contemplated. BAM₁, and BAM₂ represent different biologically active molecules, while PAA₁ and PAA₂ indicate different pharmaceutically active agents. In addition to different BAM, PAA and binding pairs that can be attached to the same POX polymer, different ratios of each of a BAM, PAA and binding pair also can be linked to the same polymer.

In some embodiments, a functionalized polyoxazoline polymer is provided, wherein said polymer comprises the formula: Iₙ—P—L—M—B, wherein I is an initiator, n≥1, P is a polyoxazoline polymer, L is a first linker comprising at least two amine groups, at least two imino (—NH—) groups or at least an amino group and an imino group, wherein said first linker is attached to said polymer by one of said at least two amine groups, at least two imino (—NH—) groups or at least an amino group and an imino group, M is a second linker and B is a bioactive material.

### BRIEF DESCRIPTION OF THE FIGURES

The following description of the figures and the respective drawings are non-limiting examples that depict various embodiments that exemplify the present invention.

FIG. 1 depicts a poly(2-ethyloxazoline) (EOx, also, EOX) (PEOX or PEOx) polymer produced through a cationic ring-opening polymerization process. The initiator, I, can comprise different functional groups, such as, an alkyl, an aryl, an ester, an amide, a sulfur-containing group, a silica-containing group, a protected amine, a protected imine and so on. n is the number of repeat units in the polymer which is dictated by reaction conditions and as a design choice.

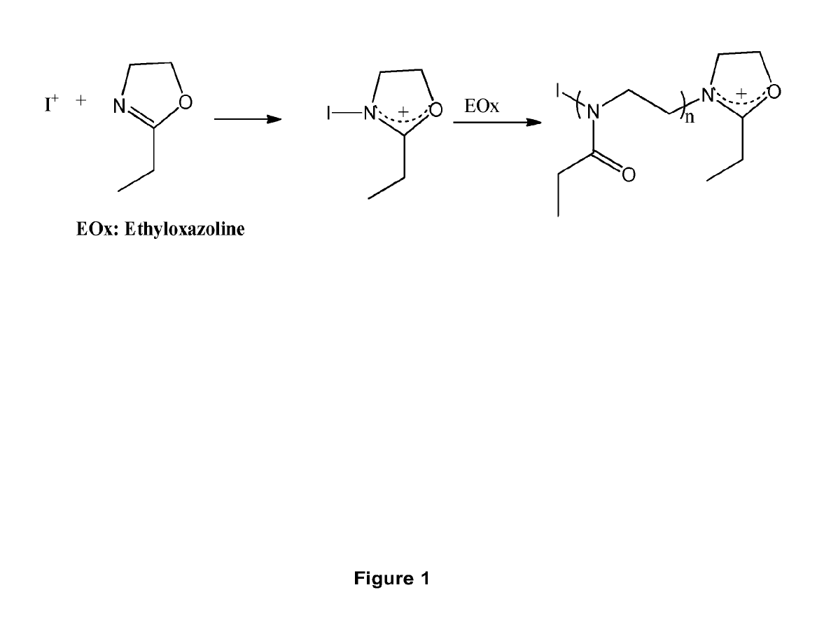

FIG. 2 depicts termination of a reactive poly (2-ethyloxazoline) polymer chain end with an excess of various small molecules with multifunctional, unprotected amino or imino groups.

FIG. 3 depicts reaction of amino-terminated or imino-terminated poly (2-ethyloxazoline) with additional linker groups, including glycidol (G), succinic anhydride (SA) or an imidazole-drug ester.

FIG. 4 depicts an example for the synthesis of a poly (2-ethyloxazoline) and camptothecin composition.

FIG. 5 depicts an example for the synthesis of a poly (2-ethyloxazoline) and gemcitabine (Gem) composition.

FIG. 6 depicts the synthesis of a modified poly (2-ethyloxazoline) polymer prior to linking with a bioactive material. MA is methacrylate. EDA is ethylenediamine. PEOx is PEOX.

FIG. 7 depicts the synthesis of a PEOX-gemcitabine composition with an S-S linkage within the PEOX polymer. The initiator is cleavable. CISO₂PhCH₃ is p-toluenesulfonyl chloride. NHS is N-hydroxysuccinimide. DCC is dicyclohexylcarbodiimide. In the second reaction step, EOX is added. In the fourth reaction step, SA is added.

FIG. 8 depicts the synthesis of an antibody(IgG)-gemcitabine composition through a PEOX polymer linker. DTT is dithiothreitol. MAL is maleimide. PEG is polyethyleneglycol.

FIG. 9 depicts comparison data on cancer cell killing rates between PEOX-camptothecin (ANP-009_C) and Irinotecan (CPT11), a semisynthetic analog of camptothecin, using MCF-7, a breast cancer cell line.

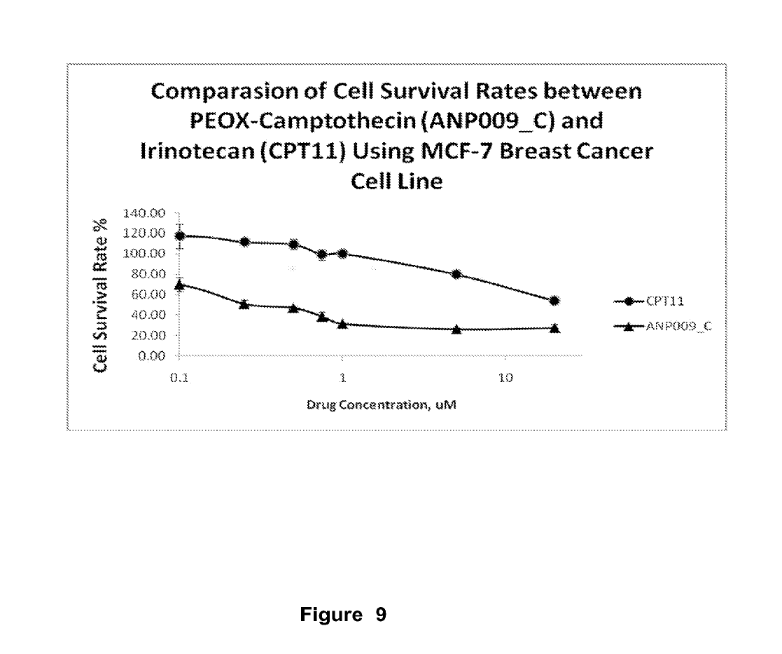

FIG. 10 depicts comparison data on cancer cell killing rates between PEOX-camptothecin (ANP-009_C) and Irinotecan (CPT11) using H460, a lung cancer cell line.

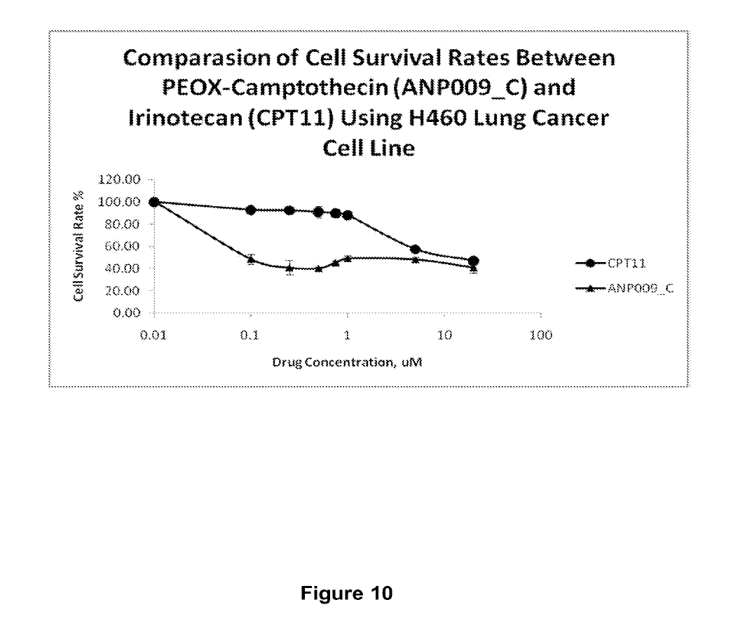

### DETAILED DESCRIPTION

Features and advantages of the present invention will be more readily understood, by those of ordinary skill in the art, from reading the following detailed description. It is to be appreciated that certain features of the invention, which are described above and below in the context of separate embodiments, may also be provided in combination in a single embodiment. Conversely, various features of the invention that are, for brevity, described in the context of a single embodiment, may also be provided separately or in any combination or sub-combination. In addition, references in the singular may also include the plural (for example, “a” and “an” may refer to one, or one or more) unless the context specifically states otherwise.

Use of numerical values in the various ranges specified in this application, unless expressly indicated otherwise, are stated as approximations as though minimum and maximum values within the stated ranges were both proceeded by the word, “about”. In this manner, slight variations above and below the stated ranges can be used to achieve substantially the same results as values within the ranges. Also, disclosure of ranges is intended as a continuous range including every value between the minimum and maximum values and including the minimum and maximum cited values.

As used herein, an “alkyl group”, comprises aliphatic compounds, which can be linear or branched chains, can comprise one or more non-aromatic rings and so on. The alkyl can comprise one or more saturated bonds or can be unsaturated. Also, the alkyl can comprise one or more pendant groups, functional groups, modifications and the like, which can include other elements, such as, oxygen, nitrogen, sulfur, iodine, bromine or chlorine, or can contain a polar group, such as hydroxyl or amine or a non-polar group, such as, a hydrocarbon, such as, an aliphatic group or an aromatic group, which also can be substituted.

As used herein, an “aryl group” comprises a hydrocarbon with alternating double and single bonds between carbons, that is, an aromatic structure. An aryl can comprise multiple aromatic rings, which may be fused or joined, and which can be substituted. An aryl also can comprise a heterocyclic compound, such as, an unsubstituted and a substituted furan or pyridine.

As used herein, an “ester” is a chemical compound derived by reacting an oxoacid, such as, a carboxyl acid, with a hydroxyl compound, such as, an alcohol or phenol. Esters are usually derived from an inorganic acid or organic acid in which at least one —OH (hydroxyl) group is replaced by an —O-alkyl (alkoxy) group, and most commonly from carboxylic acids and alcohols. That is, esters are formed by condensing an acid with an alcohol.

As used herein, an “amide” is an organic compound that contains the functional group consisting of a carbonyl group (R—C═O) linked to a nitrogen atom (N).

As used herein, a “sulfur-containing compound” is one which contains one or more reactive sulfur atoms. The compound can be 2-(p-toluenesulfonyloxy) ethyl disulfide (TOEDS), (chloromethyl) methyl disulfide or bis(iodomethyl)methyl disulfide, 2-bromoethyl disulfide, for example.

As used herein, a “silica-containing compound” is one containing at least one silicon atom. The compound can be (3-chloropropyl)triethoxysilane, (3-bromopropyl)trimethoxysilane or (3-iodopropyl)trimethoxysilane, for example.

As used herein, “protective groups for an amine or an imine” or a “protected amine or protect imine” is an amine or imine which comprises a carboxybenzyl group, a p-methoxybenzyl carbonyl group, a tert-butyloxycarbonyl group, a 9-fluorenylmethyloxycarbonyl (FMOC) group and so on.

Herein, the term B (such as used in B, B1 or B2 in polymer formulas), BM and BAM can be used as synonyms each referring to a bioactive material or a biologically active molecule and can be used interchangeably. The term B including B1, B2, BM or BAM each can independently comprise a PAA (pharmaceutically active agent) including small molecule drugs, large molecule drugs, chemotherapy drugs, immunosuppressant drugs, a combination thereof, or the like.

As known, a polymer comprises a number of component monomers which may be the same or different. As known, the molecular formula of a polymer can be denoted by naming the one or more monomers and with a subscript indicating the number of that monomer in the polymer. As used herein, “n” is not meant to relate to any particular sized polymer. Instead, n is meant to indicate a polymer and the value of n is a design choice, based, for example, on the intended use.

### Binding Pairs

A member of a binding pair can include, for example, an antibody or an antigen-binding portion thereof, an antigen, an avidin/streptavidin/neutravidin, anti-streptavidin, a biotin, a dinitrophenol (DNP), an anti-DNP antibody, a digoxin, an anti-digoxin antibody, a digoxigenin, an anti-digoxigenin antibody, a hapten, an anti-hapten antibody, a polysaccharide, a polysaccharide binding moiety, such as a lectin, a receptor, a fluorescein, an anti-fluorescein antibody, a complementary DNA, an RNA, an antibody and an Fc receptor and so on that are complementary and bind each other or one binds the other. A member of a binding pair can be included in the polymer of this invention so the polymer can bind to the other member of the binding pair when desired, such as in vivo or in vitro.

### Pharmaceutically Active Agents (PAA)-Small and Large Molecule Drugs

Small molecule drugs are defined as those with a molecular weight that can be less than about 1,000 Da (Dalton), while large molecule drugs are larger sized and also can comprise biologicals or are derived from a biological molecule. For example, a large molecule drug can include a natural biopolymer, such as, a polypeptide (e.g., a protein), a nucleic acid (e.g., DNA or RNA), a CpG oligonucleotide (ODN), a polysaccharide, a lipid and so on, as well as combinations thereof, such as, a glycolipid, a glycoprotein, a lipoprotein and so on, and can include synthetic biopolymers, such as, an aptamer, a peptide nucleic acid (PNA) and so on.

Examples of pharmaceutically active agents (PAA), such as, drugs, include, but are not limited to, chlormethine, chlorambucil, busulfan, thiotepa, cyclophosphamide, estramustine, ifosfamide, meclilorethamine, melphalan, uramustine, lonuistine, streptozotocin, dacarbazine, procarbazine, temozolainide, cisplatin, carboplatin, oxaliplatin, satraplatin, (SP-4-3)-(cis)-aminedichloro-[2-methylpyridine]-platinum (II), methotrexate, permetrexed, raltitrexed, trimetrexate, camptothecin, camptothecin derivatives (such as, irinotecan, topotecan etc.), cladribine, chlorodeoxyadenosine, clofarabine, fludarabine, mercaptopurine, pentostatin, thioguanine, azacitidine, capecitabine, cytarabine, edatrexate, floxuridine, 5-fluorouracil, gemcitabine, troxacitabine, bleomycin, dactinomycin, adriamycin, actinomycin, mithramycin, mitomycin, mitoxantrone, porfiromycin, daunorubicin, doxorubicin, liposomal doxorubicin, epirubicin, idarubicin, valrubicin, phenesterine, tamoxifen, piposulfancamptothesin, paclitaxel, docetaxel, taxotere, vinblastine, vincristine, vindesine, vinorelbine, amsacrine, etoposide, teniposide, fluoxymesterone, testolactone, bicalutamide, cyproterone, flutamide, nilutamide, aminoglutethimide, anastrozole, exemestane, formestane, letrozole, dexamethasone, prednisone, diethylstilbestrol, fulvestrant, raloxifene, toremifene, buserelin, goserelin, leuprolide, triptorelin, medroxyprogesterone acetate, megestrol acetate, levothyroxine, liothyronine, altretamine, arsenic trioxide, gallium nitrate, hydroxyurea, levamisole, mitotane, octreotide, procarbazine, suramin, thalidomide, methoxsalen, sodium porfimer, bortezomib, erlotinib hydrochloride, gefitinib, imatinib mesylate, semaxanib, adapalene, bexarotene, trans-retinoic acid, 9-cis-retinoic acid, N-(4-hydroxyphenyl) retinamide, tiuxetan, ozogamicin, glargine and so on, as well as derivatives thereof.

Large molecule drugs include, for example, proteins, such as, enzymes, such as, L-asparaginase, antibodies and antigen-binding portions thereof, such as, abciximab, adalimumab, alefacept, alemtuzumab, alirocumab, apatinib, arcitumomab, atezolizumab, avelumab, basiliximab, belimumab, besilesomab, bevacizumab, bezlotoxumab, brentuximab, brodalumab, canakinumab, capromab, catumaxomab, certolizumab, cetuximab, daclizumab, daratumumab, denosumab, dinutuximab, dupilumab, durvalumab, eculizumab, efalizumab, efalizumab, elotuzumab, evolocumab, fanolesomab, gemtuzumab, golimumab, herceptin adc, ibritumomab, ibritumomab tiuxetan, idarucizumab, imiciromab, inflectra, infliximab, ipilimumab, ixekizumab, lifirafenib, mepolizumab, muromonab-cd3,natalizumab, necitumumab, nivolumab, nofetumomab, obiltoxaximab, obinutuzumab, ocrelizumab, ofatumumab, olaratumab, omalizumab, osimertinib, palivizumab, pamiparib, panitumumab, pembrolizumab, pertuzumab, pyrotinib, ramucirumab, ranibizumab, raxibacumab, reslizumab, rituximab, rituximab, satumomab, secukinumab, shr1210m, shr-a1201,siltuximab, sulesomab, tislelizumab, tocilizumab, tositumomab, trastuzumab, ustekinumab, vedolizumab, votumumab, and zanubrutinib; cytokines, such as, interleukins, interferon α2a, interferon α and granulocyte colony stimulating factor (GCSF), peptide hormones, such as, insulin, glucagon, glucagon like peptide-1, erythropoietin, follicle stimulating hormone and so on, ligands of cell surface receptors, lectins, nucleic acids, such as siRNA's, ribozymes, antisense nucleic acids, naked nucleic acids, CpG oligonucleotide (ODN) and so on, viruses, virus-like particles and the like. Examples include Ecallantide.

Pharmaceutically Active Agents (PAA) can also comprise one or more chemotherapy drugs such as 5-FU (Fluorouracil), Abemaciclib, Abiraterone Acetate, Abitrexate (Methotrexate), Abraxane (Paclitaxel Albumin-stabilized Nanoparticle Formulation), Acalabrutinib, Adcetris (Brentuximab Vedotin), Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), Becenum (Carmustine), Beleodaq (Belinostat), Belinostat, Bendamustine Hydrochloride, BEP, Besponsa (Inotuzumab Ozogamicin) , Bevacizumab, Bexarotene, Bexxar (Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, Calquence (Acalabrutinib), Campath (Alemtuzumab), Camptosar (Irinotecan Hydrochloride), Capecitabine, Carac (Fluorouracil-Topical), Carboplatin, CARBOPLATIN-TAXOL, Carfilzomib, Carmubris (Carmustine), Carmustine, Casodex (Bicalutamide), Ceritinib, Cerubidine (Daunorubicin Hydrochloride), Cervarix (Recombinant HPV Bivalent Vaccine), Cetuximab, Chlorambucil, CHLORAMBUCIL-PREDNISONE, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, Cosmegen (Dactinomycin), Cotellic (Cobimetinib), Crizotinib, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, Fareston (Toremifene), Farydak (Panobinostat), Faslodex (Fulvestrant), Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, HPV Nonavalent Vaccine, HPV Quadrivalent Vaccine, Hycamtin (Topotecan Hydrochloride), Hydrea (Hydroxyurea), Hydroxyurea, Ibrance (Palbociclib), Ibritumomab Tiuxetan, Ibrutinib, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Interleukin-2 (Aldesleukin), Intron A (Recombinant Interferon Alfa-2b), Iodine I131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), Ofatumumab, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), Osimertinib, Oxaliplatin, Paclitaxel, Paclitaxel Albumin-stabilized Nanoparticle Formulation, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), Revlimid (Lenalidomide), Rheumatrex (Methotrexate), Ribociclib, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, Valrubicin, Valstar (Valrubicin), VAMP, Vandetanib, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), XELIRI, Xeloda (Capecitabine), XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate), or a combination thereof.

The B including B1, B2, BM, BAM or PAA (pharmaceutically active agent) used herein each can also comprise at least one immunosuppressant. The immunosuppressant can be used as the B1, B2 or both B1 and B2.Immunosuppressant, also known as immunosuppressive drug or immunosuppressive agent, can include corticosteroids comprising prednisone (Deltasone, Orasone), budesonide (Entocort EC) or prednisolone (Millipred); calcineurin inhibitors comprising cyclosporine (Neoral, Sandimmune, SangCya) or tacrolimus (Astagraf XL, Envarsus XR, Prograf); mTOR inhibitors comprising sirolimus (Rapamune, Rapamycin) or everolimus (Afinitor, Zortress); IMDH inhibitors comprising azathioprine (Azasan, Imuran), leflunomide (Arava) or mycophenolate (CellCept, Myfortic); biological drugs including monoclonal antibodies comprising abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta) or muromonab (Orthoclone OKT3).

Additional examples of PAA include recombinant blood factors, such as, Factor III, antihemophilic factor, Factor VIII, antithrombin, thrombin, Factor Vila, Factor IX; tissue plasminogen activator, such as, TNK-tPA, tenecteplase and alteplase, including truncated forms thereof, such as, reteplase, hirudin, protein C and so on; recombinant hormones, such as, insulin, such as, insulin detemir, a long-acting insulin analog, insulin glulisine, a rapid-acting insulin analog and insulin glargine (another long-acting insulin analog); human growth hormone, also known as somatropin, follicle-stimulating hormone, such as, the .alpha. subunit thereof, such as, corifollitropin α, glucagon like peptide-1, parathyroid hormone, and truncated forms thereof, such as, terpiparatide, B-type natriuretic peptide, calcitonin, luteinizing hormone, hCG, TSH, glucagon and so on; recombinant growth factors, such as, erythropoietin, such as, epoetin θ, erythropoietin α. and epoetin ζ, long acting analogs thereof, such as, darbepoetin α; colony stimulating factors, such as, GM-CSF and G-CSF, insulin-like growth factor (IGF), a complex of IGF and IGF binding proteins, such as, mecasermin rinfabate, keratinocyte growth factor, platelet-derived growth factor and so on; recombinant cytokines, such as, interferons and interleukins, such as, interferon α, IFN-a-2b, interferon β, interferon-β-1B, IFN-β-1a, IL-11, IL-2, IFN-γ1b and so on; recombinant vaccines, such as those for Hepatitis B, papillomavirus (HPV), cholera toxin B subunit, OspA, a lipoprotein found on the surface of B. burgdorferi), pertussis toxin and so on; monoclonal antibody and antigen-binding portions thereof, made to any antigenic entity as known in the art, such as, denosumab, tocilizurmab, besilesomab, ofatumumab, canakinumab, catumaxomab, golimumab, steknumab, ranibizumab, eculizumab, panitumumab, natalizumab, omalizumab, ibritumonmab, cetuximab, efalizumab, adalimumab, tositumomab, infliximab, palivizumab, daclizumab, votumumab, basiliximab, sulesomab, igovomab, abciximab and so on; other recombinant biologics, such as, bone morphogenetic proteins, such as, BMP-7 and BMP-2) and so on; recombinant enzymes, such as, α glucosidase, glucocerebrosidase, iduronate-2-sulfatase, N-acetylgalactosidase, 4-sulfatase, β-glucocerebrosidase, DNase, hyaluronidase, α-galactosidase, α-L-iduronidase, urate oxidase and so on; oligonucleopeptides; and so on, as well as combinations thereof, such as, rilonacept (a dimeric fusion protein of the extracellular (EC) domain of the IL-1 receptor and the Fc portion of an IL-1 IgG-1), romiplostim (a dimeric fusion protein with each monomer consisting of two thrombopoietin receptor-binding domains and the Fc region of an IgG-1), Abatacept (an immunoglobulin fused to the EC domain of CTLA-4), alefacept (containing the Fc portion of an antibody and a portion of CFA-3) and so on.

### Biologically Active Molecules (BAM)

In addition to binding pairs, certain large molecule drugs and PAA's described above, some additional examples of a BAM include, but are not limited to, interleukins, interferons, CD4 and other CD molecules, including agonists and antagonists thereof, Fc receptor, acetylcholine receptor (AChR), T cell receptor, hormone receptors, such as, an insulin receptor, tumor necrosis factor, granulocyte-macrophage colony stimulating factor, antibodies and antigen-binding fragments thereof, such as, Fab and scAb, phage, phage fragments, sugars containing sialic acid residues, cell targeting peptides, DNA fragments, RNA fragments, hormones, such as, insulin and hCG, enzymes, sialic acid, polysaccharides, lectin, porphyrins, nucleotides, viruses, viral fragments and so on, other receptors and the like.

### Bioactive Materials (BM)

In the disclosure, bioactive materials comprise binding pairs, a BAM, a PAA, small and large molecule drugs and any other biologically active or related molecules.

### Polyoxazoline-Bioactive Material Composition

POX-BM compositions of interest can have the following formula:

Iₙ—P—L—M—B,

wherein I is an initiator moiety and n≥1. I can comprise an alkyl, an aryl, an ester, an amide, a sulfur-containing compound, a silica-containing compound, a protected amine or a protected imine;

P is a POX polymer;

L is a linker comprising at least two amine groups, at least two imino (—NH—) groups or at least an amino group and an imino group, wherein said first linker is attached to said polymer by one of said at least two amine groups, at least two imino (—NH—) groups or at least an amino group and an imino group, in embodiments, said linker can comprise a small molecule comprising plural amino or imino (—NH—) groups, wherein said small molecule can comprise either an entire or a portion of ammonia, ethylenediamine (EDA), piperazine, 1,4,7,10-tetraazacyclododecane (cyclen), tris(2-amino ethyl) amine (tren), 4-(aminomethyl) piperidine, hexamethylenediamine, 1,3-diaminopropane, triethylenetetramine, 2,2′-(ethylenedioxy)bis(ethylamine), 1,11-diamino-3,6,9-trioxaundecane, diethylenetriamine, tris(2-aminoethyl)amine, 1,8-diaminooctane and so on; and

M is an additional linker, comprising, for example, a CDI or a partial CDI-functionalized molecule (e.g., an imidazole-PAA ester), glycidol, succinic anhydride, acrylic ester, amidoamine, linear or branched polyamidoamine, acrylamide and/or a heterofunctional molecule, with one end of the heterobifunctional molecule activated with, for example, N-hydroxysuccinimide (NHS) or an aldehyde functional group, for reaction with the L linker. The other end of the heterobifunctional molecule comprises a reactive group to facilitate reaction with the B entity, such as, a —OH, —COON, —COONa, an ester, an amide, a maleimide or an —H group. Alternatively, B also can be physically mixed with Iₙ—P—L—M to form a polyoxazoline-bioactive material composition without the need for a specific reaction to form a covalent bond between B and the remainder of the composition.

A partial CDI-functionalized molecule comprises a reaction product between a CDI and, for example, an OH-functionalized molecule or a bioactive material (BM).

B is a bioactive material linked with said polymer through either a hydrolytically stable or an unstable linkage. Alternatively, in embodiments, B may be associated with the remainder of a composition of interest in the absence of a formal linkage as described herein. Hence, mere mixing of B with I-P-L-M yields a composition of interest.

Therefore, I, L and M can possess the same or different functional groups. When possessing different functional groups, one set of BM's can be attached at the L end, while a different set of BM's can be linked to an initiator end. For example, one can link a small molecule drug moiety at the L end while an antibody can be attached to the initiator, I, end(s) to form an antibody/drug conjugate (ADC). Alternatively, an antibody can be attached at the L end while the small drug molecule can be linked to the initiator I end(s) to also form an ADC at the same or at different ratios as the previous method. Other combinations may include linking two different BM molecules or two different PAA's at the I and L ends, respectively.

In addition, the linkers can be tailored to produce either hydrolytically stable or unstable chemical linkages so that various delivery systems can be generated, depending on the need for controllable B release rates. Readily cleavable linkages include, but are not limited to, an anhydride bond, an S—S (sulfur-sulfur bond) linkage, a peptide bond that can be cleaved by an enzyme and so on.

In another aspect of the invention, the POX can be linear, star branched, comb branched, dendritically branched or a randomly branched polymer. Said branched or dendritic polymers can either be symmetrically or asymmetrically branched.

In another aspect of the invention, the POX can be a poly (unsubstituted oxazoline) or a poly (substituted oxazoline) polymer. The poly (substituted oxazoline) can be poly (2-methyloxazoline), poly (2-ethyloxazoline), poly (2-propyloxazoline) or poly (2-butyloxazoline).

In a further aspect of this invention, a functionalized polymer of this invention can comprise a polyoxazoline polymer P reacted with at least one initiator I and a first linker L, wherein the L is further reacted with two or more second linker M, and wherein,

the initiator I comprises a first functional group that is reacted with P and a second functional group that is reactive with a first biologically active molecule B1;

the first linker I comprises three or more active hydrogens in amine groups or compound selected from an NH₃, two or more —NH₂ groups, three or more imino (—NH—) groups, or a combination of —NH₂ and (—NH—) groups, wherein the first linker L is reacted with the P with one of the active hydrogens;

the M comprises a carbodiimide (CDI), a CDI-functionalized molecule, glycidol (G), succinic anhydride (SA), acrylic ester, acrylamide or any of the aforementioned heterofunctional molecule, the M comprises at least one first linker group reacted with one of the active hydrogens of the L and at least one second linker group reactive with a second biologically active molecule B2.

The second linker M can further comprise a reaction product of compounds that have functional group —NH₂, —NH, OH, COOH, an ester, an amide, a maleimide, acid halide, acryl halide, alkyl halide, allyl halide, benzyl halide, acrylate, methacrylate, isocyanate, isothiocyanate, ketone, aldehyde, epoxide, —SH or a combination thereof. In one example, M can comprise a reaction product of ethylenediamine (EDA) and methacrylate (MA).

For simplicity, the first linker L, second linker M including individual molecules described in M such as CDI, G, SA, etc., initiator I, polyoxazoline polymer P, biologically active molecule B1 and B2 each can represent a stand-alone molecule prior to incorporating into a polymer or a reacted residue of the molecule in a polymer. For example, a first linker L can represent a stand-alone ethylenediamine (EDA) H₂N(CH₂)₂NH₂ having 4 active hydrogens in two —NH₂ groups prior to incorporating into a polymer, an EDA in a polymer wherein one of the active hydrogens is reacted with the polymer leaving three active hydrogens in —NH and —NH₂ groups, an EDA in a polymer wherein one of the active hydrogens is reacted with the polymer and two active hydrogens reacted with two second linker M leaving one active hydrogen in the -NH group, or an EDA reacted with the polymer and three second linker M with no further active hydrogens in the NH groups, unless specifically defined otherwise. When specifically defined, the term “reacted I”, “reacted P”, “reacted L”, “reacted M”, “reacted B1” or “reacted B2” refers to a molecule reacted with other molecule(s) with reactive groups suitable for reactions between molecules. The term “reactive” used in relation to a functional group means a functional group that can react with a certain other groups, but is not reacted with that other group yet.

The functionalized polymer can comprise a formula (1):

Iₙ—P—L—(M)_{y} (1)

wherein, n and y each is an integer, n≥1, and

y≥2.

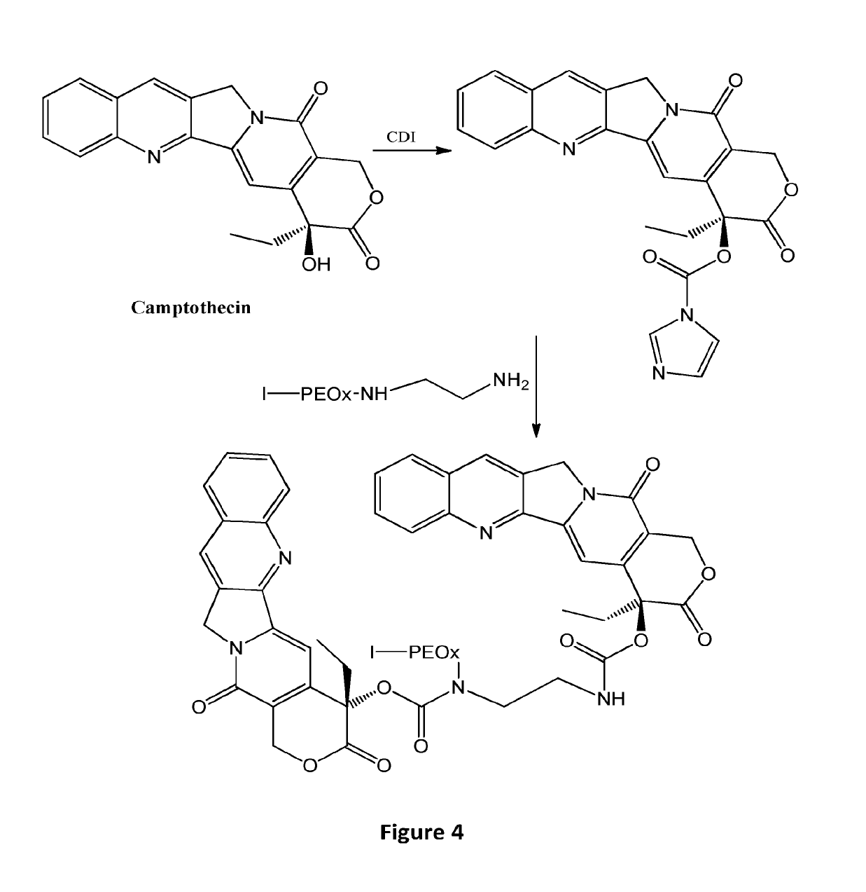

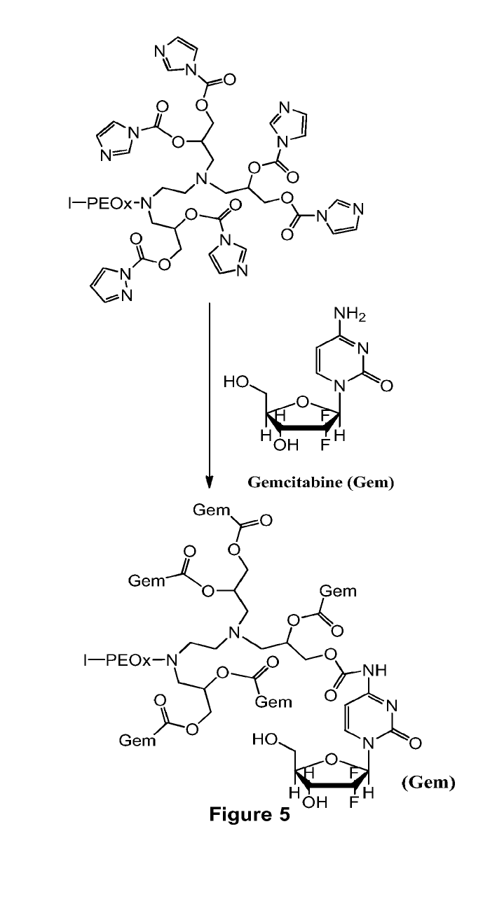

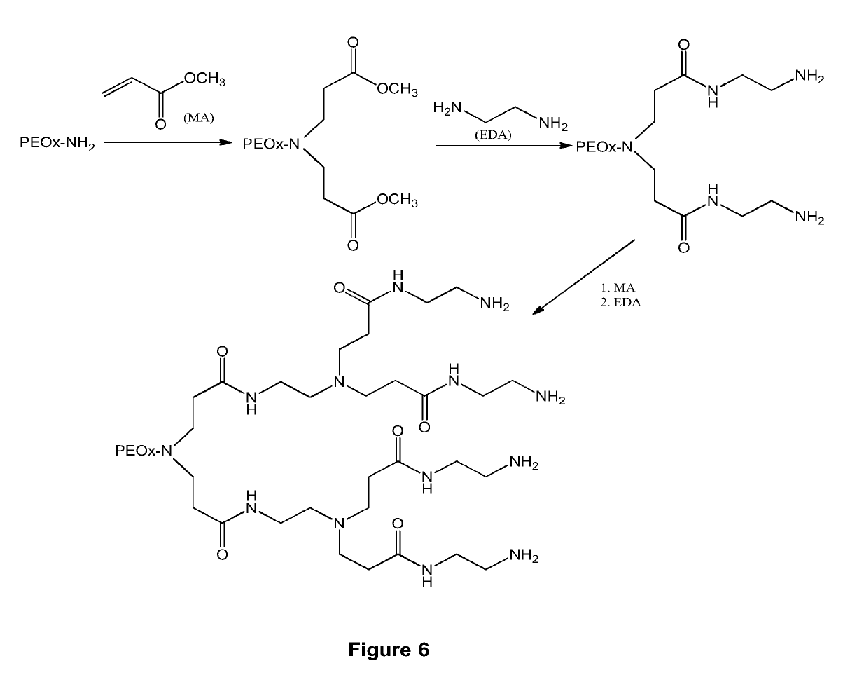

Any of aforementioned polymers and initiators can be suitable. The second linker M is each reacted to with one of the active hydrogens of the first linker L. In one example, L can be an EDA reacted with polymer P and having 3 active hydrogens in a polymer PEOX-NH(CH₂)₂NH₂, wherein two of the 3 active hydrogens can be reacted to a second linker M (CDI), such as those shown in FIG. 4. In one example, L can be an EDA having 3 active hydrogens in a polymer PEOX-NH(CH₂)₂NH₂ and all 3 active hydrogens can be reacted to a second linker M, such as those shown in FIG. 5. Each M can have one or more second linker group reactive with a second biologically active molecule B2. In one example, M can have three second linker groups each reacted with B2 and one additional reactive second linker group, such as shown in FIG. 4. In another example, each M can have two reactive second linker groups that each can be subsequently reacted to B2 (Gem), such as shown in FIG. 5. In yet another example, M can have 4 or more reactive second linker groups, such as shown in FIG. 6.

The functionalized polymer of formula (1) can be used as a polymer product or as an intermediate for producing further functionalized polymers described herein.

The functionalized polymer disclosed herein can further comprise at least one first biologically active molecule B1 reacted with the initiator I, at least one second biologically active molecule B2 reacted with at least one of the M, or a combination thereof. In one example, a functionalized polymer comprises B1 reacted with the initiator I in the polymer producing a polymer of a formula B1 —Iₙ—P—L—(M)_{y}. In another example, a functionalized polymer comprises at least one B2 reacted with at least one second linker M in the polymer. In yet another example, a functionalized polymer comprises a formula Iₙ —P—L—(M—(B2)_{z})_{y}, y and z each is an integer and y≥2, z≥0. In yet another example, a functionalized polymer comprises both B1 and B2 reacted with I and M, respectively. Each M can be independently reacted to one or more B2 when B2 is present.

When each M is reacted with a same number of B2 or the lack thereof, the functionalized polymer can comprise a formula (2):

(B1)ₓ—Iₙ—P—L—(M—(B2)_{z})_{y}

or a formula (3):

((B1)ₓ—I)ₙ—P—L—(M—(B2)_{z})_{y}

n, x, y and z each is an integer and

x≥0,

n≥1,

y≥2,

z≥0, with a proviso that one of x or z is not 0.

When x and z both are 0, formula (2) and (3) are the same as formula (1). The value of x can be dependent upon the number of second functional groups in the initiator I. When an initiator I has one second functional group, x can be from 0 up to n. When an initiator I has multiple second functional groups, such as 2, 3 or 4 second functional groups, x can be from 0 to 2, 3, or 4 for each of the initiator, respectively.

The value of z can be dependent upon the number of second linker groups in the second linker M. When each M has one second linker group, z can be from 0 up to 1. When an M has multiple second linker groups, such as 2-8 second linker groups, z can be from 0 to 2-8, respectively.

The functionalized polymer can also comprise a formula (4):

(B1)ₓ—Iₙ—P—L—(M)_{y}—(B2)_{z}

or a formula (5):

((B1)ₓ—I)ₙ—P—L—(M)_{y}—(B2)_{z}

with aforementioned n, x, y and z.

The B1 or B2 each can independently comprise a member of a binding pair (BP), a bioactive material (BM), a biologically active molecule (BAM), or a pharmaceutically active agent (PAA). Any of the aforementioned BP, BAM and PAA can be suitable. In one example, a functionalized polymer of this invention can comprise a B1 comprising a member of a binding pair (BP), a bioactive material (BM), a biologically active molecule (BAM), or a pharmaceutically active agent (PAA). In another example, a functionalized polymer of this invention can comprise a B2 comprising a member of a binding pair (BP), a biologically active molecule (BAM), or a pharmaceutically active agent (PAA). In yet another example, a functionalized polymer of this invention can comprise both B1 and B2 each comprising a member of a binding pair (BP), a biologically active molecule (BAM), or a pharmaceutically active agent (PAA).

The B1 or B2 each independently can comprise a small molecule drug, a large molecule drug, a chemotherapy drug, an immunosuppressant drug, or a combination thereof. As disclosed herein, each of the B1 and B2 can also comprise a targeting moiety.

The large molecule drug can comprise a polypeptide, a polynucleotide, an antibody, a monoclonal antibody (mAb) such as the aforementioned monoclonal antibodies or a combination thereof. The small molecule drug can comprise a taxane, a paclitaxel, a gemcitabine, a camptothecin, anthracyclines, doxorubicin, epirubicin, cisplatin, carboplatin, vinorelbine, capecitabine, ixabepilone, eribulin or a combination thereof.

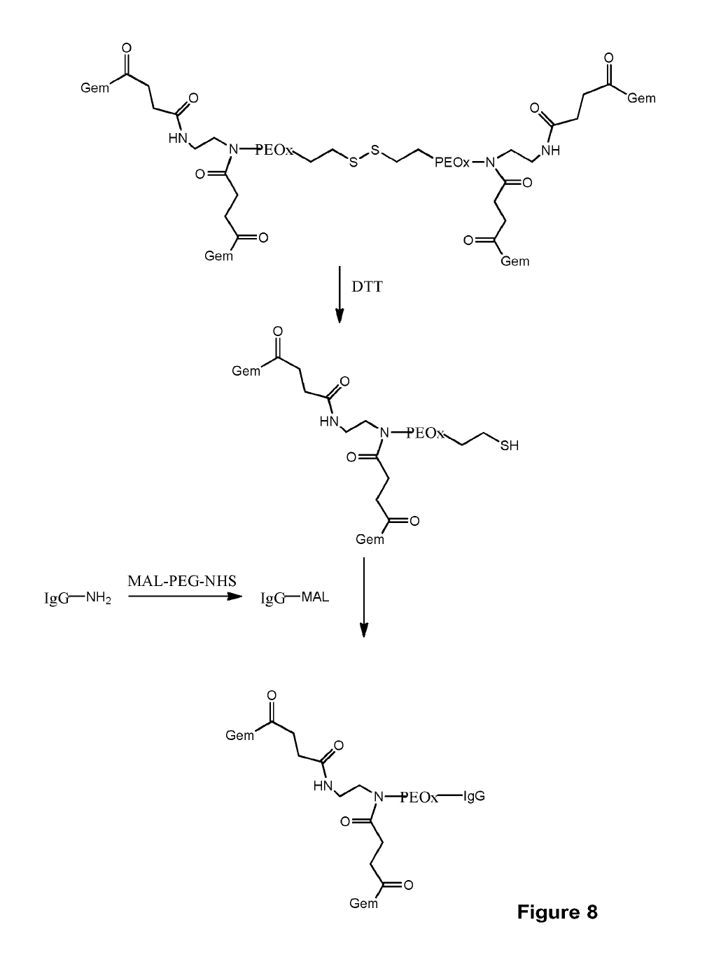

For a functionalized polymer of this invention, the B1 can be a monoclonal antibody selected from abciximab, adalimumab, alefacept, alemtuzumab, alirocumab, apatinib, arcitumomab, atezolizumab, avelumab, basiliximab, belimumab, besilesomab, bevacizumab, bezlotoxumab, brentuximab, brodalumab, canakinumab, capromab, catumaxomab, certolizumab, cetuximab, daclizumab, daratumumab, denosumab, dinutuximab, dupilumab, durvalumab, eculizumab, efalizumab, efalizumab, elotuzumab, evolocumab, fanolesomab, gemtuzumab, golimumab, herceptin adc, ibritumomab, ibritumomab tiuxetan, idarucizumab, imiciromab, inflectra, infliximab, ipilimumab, ixekizumab, lifirafenib, mepolizumab, muromonab-cd3, natalizumab, necitumumab, nivolumab, nofetumomab, obiltoxaximab, obinutuzumab, ocrelizumab, ofatumumab, olaratumab, omalizumab, osimertinib, palivizumab, pamiparib, panitumumab, pembrolizumab, pertuzumab, pyrotinib, ramucirumab, ranibizumab, raxibacumab, reslizumab, rituximab, rituximab, satumomab, secukinumab, shr1210m, shr-a1201,siltuximab, sulesomab, tislelizumab, tocilizumab, tositumomab, trastuzumab, ustekinumab, vedolizumab, votumumab, or zanubrutinib; a cytokine selected from an interleukin, interferon α2a or interferon α; a hormone selected from granulocyte colony stimulating factor (GCSF), insulin, glucagon, glucagon like peptide-1, erythropoietin or follicle stimulating hormone; a ligand of cell surface receptors; a lectin; nucleic acids selected from an siRNA, a ribozyme, antisense nucleic acids, naked nucleic acids, a CpG oligonucleotide (ODN); a virus; or a virus-like particle; and the B2 comprises an aforementioned chemotherapy drug or a small molecule drug comprising. In one example, B2 comprises aforementioned small molecule drug having a molecular weight less than about 1,000 Da (Dalton). In another example, B2 comprises chemotherapy drug. In yet another example, B2 comprises a taxane including paclitaxel such as paclitaxel (Taxol), docetaxel (Taxotere), and albumin-bound paclitaxel (Abraxane); a gemcitabine (Gemzar); a camptothecin; anthracyclines including doxorubicin, pegylated liposomal doxorubicin and Epirubicin; Platinum agents including cisplatin and carboplatin; Vinorelbine (Navelbine); Capecitabine (Xeloda); ixabepilone (Ixempra); eribulin (Halaven); or a combination thereof. The processes disclosed herein including the schematic outline exemplified in FIG. 8 can be suitable for producing a functionalized polymer having both an mAb and a small molecule drug.

For the functionalized polymer disclosed herein, polymer P (also referred to as “P”, throughout this disclosure) can comprise a poly(unsubstituted oxazoline), a poly(substituted oxazoline), or a combination thereof. The poly(substituted oxazoline) can comprise a poly(2 methyloxazoline), a poly(2-ethyloxazoline), a poly(2-propyloxazoline), a poly(2 butyloxazoline) or a combination thereof. The polymer P can be a linear polymer, a dendritic polymer, a randomly branched polymer, symmetrically branched polymer, asymmetrically branched polymer, or a combination thereof.

In functionalized polymer disclosed herein, L can comprise a reacted NH3, ethylenediamine, tris(2 aminoethyl)amine, 4 (aminomethyl)piperidine, 1,3-diaminopropane, 2,2′ (ethylenedioxy)bis(ethylamine), diethylenetriamine, 1,4,7,10-tetraazacyclododecane, hexamethylenediamine, triethylenetetramine, or 1,8-diaminooctane.

The first linker group of the second linker M can comprise alkyl halide, alkyl sulfonate, aldehyde, ketone, ester, activated ester, acid halide, anhydride, epoxide, Isothiocyanate, acrylate, methacrylate, sulfonyl chloride, nitrous acid or a combination thereof. The second linker group of the second linker M can comprise —NH₂, —NH, OH, COOH, an ester, an amide, a maleimide, acid halide, acryl halide, alkyl halide, allyl halide, benzyl halide, acrylate, methacrylate, isocyanate, isothiocyanate, ketone, aldehyde, epoxide, —SH or a combination thereof.

The initiator I can comprise an alkyl, an aryl, an ester, an amide, a sulfur-containing compound, a silica-containing compound, a protected amine or a protected imine containing compounds. The initiator I comprises a first functional group that is reactive with a monomer to form the polymer P. The first functional group can include sulfonic acid, inorganic acid, organic acid, alkyl halide, allyl halide, benzyl halide, tosylate, p-alkyl toxylate or p-toluenesulfonic acid. The initiator further comprises a second functional group that is reactive with the first biologically active molecule B1. The second functional group can comprise an ester group, an amide group, a maleimide group, a ketone group, an aldehyde group, a —SH group, a thiolated group comrpising an —S—S— (disulfide) containing group or other sulfur-containing groups, a benzyl group, a toluensulfonyl group, a protected amino group, a carboxybenzyl group, a p-methoxybenzyl carbonyl group, a tert-butyloxycarbonyl group, a 9-fluorenylmethyloxycarbonyl (FMOC) group, a silicon containing group, or a combination thereof. Aforementioned sulfur-containing compounds, such as, 2-(p-toluenesulfonyloxy)ethyl disulfide (TOEDS), (chloromethyl) methyl disulfide, bis(iodomethyl)methyl disulfide and 2-bromoethyl disulfide, can be suitable.

The initiator I can comprise ethyl bromoacetate, methyl bromoacetate, bromoacetone, tert-butyl bromoacetate, propyl bromoacetate, benzyl bromoacetate, a silicon-containing compound and a protected amine or imine.

If the POX polymer is linear, B1 can be attached to the initiator I in the polymer reacted with the second functional group, as illustrated by Formula (2). If the POX is branched, multiple BM molecules B1 can be attached to the same polymer at the multiple initiator ends that each comprises a second functional group as illustrated in Formula (3).

The second functional group at the initiator I can be protected during polymerization with the initiator and oxazoline to form the polymer. The protected group can be deprotected to generate the second functional group that is reactive with B1. In addition, due to the stepwise reaction approach, not only the same but different bioactive molecules can be attached to the same POX polymer at the initiator I and reactive chain ends via the second linker group of the second linker M. Such a differentiated POX can be useful in linking different kinds of BM molecules, and at varying ratios.

Functional groups that can be introduced at the initiator I of the POX polymer as a second functional group or at the second linker M of the same POX polymer as a second linker group can include, but are not limited to, ethyl bromoacetate, methyl bromoacetate, bromoacetone, tert-butyl bromoacetate, propyl bromoacetate, benzyl bromoacetate, sulfur-containing compounds, such as, 2-(p-toluenesulfonyloxy)ethyl disulfide (TOEDS), (chloromethyl) methyl disulfide, bis(iodomethyl)methyl disulfide and 2-bromoethyl disulfide, silicon-containing compounds, such as, (3-chloropropyl) triethoxysilane, (3-bromopropyl)trimethoxysilane and (3-iodopropyl) trimethoxysilane and protected groups for amines or imines, such as, those comprising a carboxybenzyl group, a p-methoxybenzyl carbonyl group, a tert-butyloxycarbonyl group or a 9-fluorenylmethyloxycarbonyl (FMOC) group; and so on.

For example, TOEDS, a difunctional initiator, can be utilized to initiate the polymerization of oxazoline monomers at both ends. On termination, for example, with a large excess of ethylene diamine (EDA), a POX polymer with amino and imino functional groups at both chain ends can be produced. The chain ends further can be linked with any of a plurality of bioactive materials (BM), such as, small molecule drugs, such as, gemcitabine, camptothecin, paclitaxel and so on, either directly or indirectly through covalent linkages. When the reagents comprise a disulfide bond, addition of a reducing agent, such as, dithiothreitol (DTT), can cleave that bond to generate a sulfhydryl-functionalized POX polymer-bioactive material composition. The sulfhydryl group then can be linked, for example, with a maleimide-functionalized targeting molecule, such as, peptide, protein, such as, antibody, sialic acid, one member of a binding pair and so on to provide a differentiated POX polymer composition with one end linked with any of a plurality of bioactive materials, such as, small molecule drugs, and the other end linked with at least a targeting molecule, such as, peptide, protein, such as, antibody, sialic acid and so on. Various differentiated POX compositions, including, but not limited to, biologically active molecules to generate, such as, BAM-POX-PAA, BAM₁-POX-BAM₂, PAA₁-POX-PAA₂ and so on are contemplated. BAM₁ and BAM₂ represent different biologically active molecules, while PAA, and PAA₂ indicate different pharmaceutically active agents. In addition to different BAM, PAA and binding pairs that can be attached to the same POX polymer, different ratios of each of a BAM, PAA and binding pair also can be linked to the same polymer.

In the functionalized polymer disclosed herein, the B1 or B2 each can comprise a targeting moiety. The targeting moiety can comprise an antibody or an antigen-binding portion thereof, nucleic acids or a CpG ODN. The B2 can be linked to M through a cleavable linkage. In one example, the B2 is linked to M through an anhydride bond or a peptide bond.

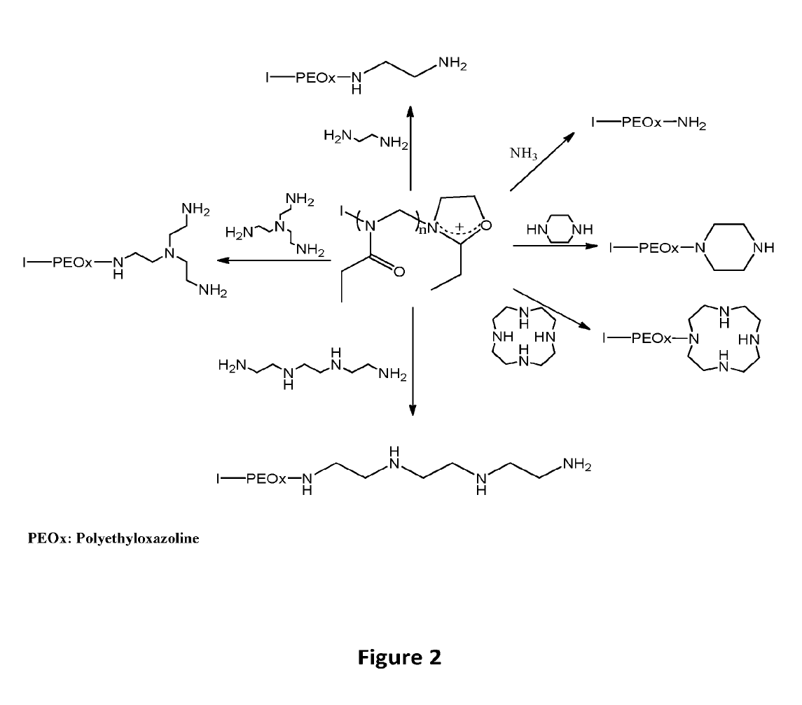

In one example of a functionalized polymer of this invention, the L comprises a NH₃ having 3 active hydrogens, wherein one of the active hydrogens is reacted with the P and the remaining active hydrogens are reacted to 2 M. For simplicity, the first linker L represents a NH₃ prior to react to the polymer and also represent the —NH₂ that is reacted to the polymer. In another example of a functionalized polymer of this invention, the L comprises at least two —NH₂ groups having 4 or more active hydrogens, wherein one of the active hydrogens is reacted with the P and the remaining active hydrogens are reacted to 2 or more M. In yet another example of a functionalized polymer of this invention, the L comprises at least one —NH₂ group and at least one —NH group having 3 or more active hydrogens, wherein one of the active hydrogens is reacted with the P and the remaining active hydrogens are reacted to 2 or more M. In yet another example of a functionalized polymer of this invention, the L comprises at least three —NH groups having 3 or more active hydrogens, wherein one of the active hydrogens is reacted with the P and the remaining active hydrogens are reacted to 2 or more M. See at least FIG. 2-FIG. 8.

Further examples of a variety of first linker L is schematically shown in FIG. 2, wherein each of the L is reacted with P via one of active hydrogens and each of L has additional 1 to 5 active hydrogens that are reactive with multiple M. The L having 2 and more active hydrogens are suitable for this invention.

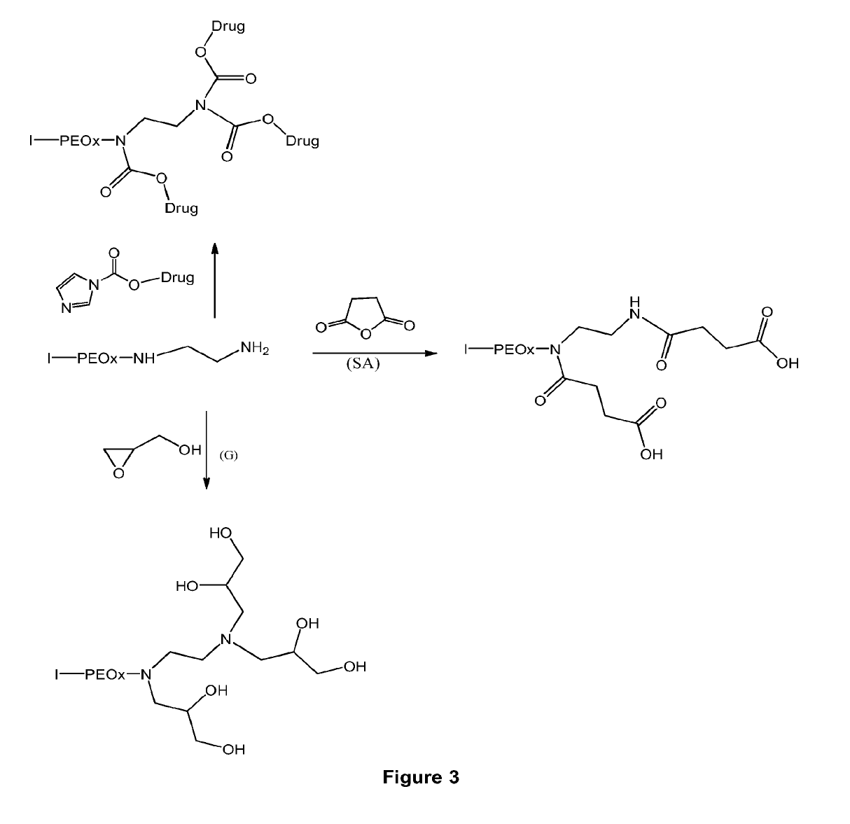

FIG. 3 exemplifies an EDA as a first linker L that is reacted with P and 2-3 M, prior to react with B2. Alternatively, a second linker M can first react with a biologically active molecule, such as a drug, then react with P. FIG. 4 exemplifies an EDA as a first linker L reacted with P and 2 M (CDI) each is reacted with B2 camptothecin. FIG. 5 exemplifies an EDA as a first linker L that is reacted with P and 3 M each reacted to 2 B2 (gemcitabine). FIG. 6 exemplifies a NH₃ as a first linker L that is reacted with P and 2 second linker M, wherein each M is a reaction product of EDA and methacrylate (MA) having multiple functional groups that can react with multiple B2, such as up to 8 units of B2 (two M each having 2 —NH₂ groups that each can react with up to 4 units of B2, i.e., z=4 and y=2 in Formula (2)).

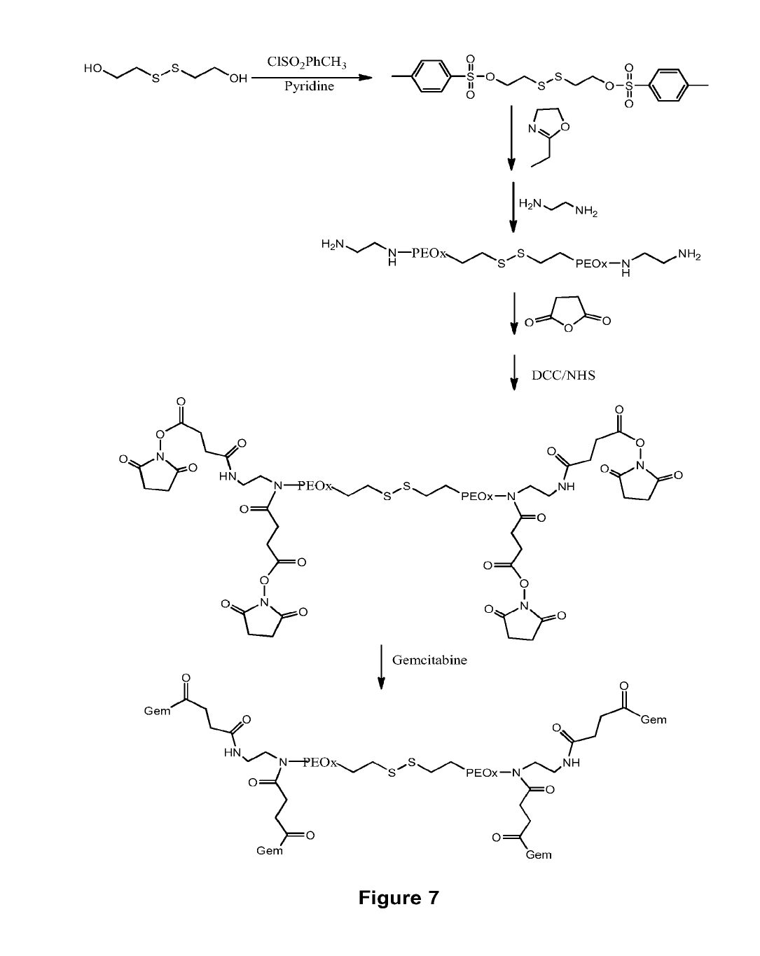

FIG. 7-FIG. 8 exemplify a cleavable linkage and one approach for attaching a targeting moiety or a B1, such as an antibody to form a functionalized polymer of this invention, together with one or more B2, such as multiple molecules of a small molecule drug.

In another aspect of the invention, the polymer-bioactive material composition can be used for various assay and drug delivery applications.

In another aspect of the invention, the differentiated, that is, comprises a heterofunctional group, POX polymers can be used to produce antibody-BM compositions for assays.

In another aspect of the invention, the differentiated/heterofunctional POX polymers can be used to produce antibody-FAA compositions for targeted drug delivery.

Also, polymer associated with multiple units of BM, and each with different properties and activities, can be used, for example, for targeting or for bridging biological entities, such as, a hormone and a receptor, or two cells. Such compositions may be formulated with acceptable carriers, diluents and additives for use, for example, in biodetection, diagnostics and therapeutics, as known in the medical, environmental, agricultural and physical sciences.

In another aspect of the invention, the said POX polymer can be modified with at least one monomer capable of forming additional branches at a given time so that new material properties can be achieved, wherein the said modified polymer is defined as a modified POX polymer. A suitable monomer can be one carrying plural reactive functional groups, which can be the same or different.

The molecular weight of said polymers can range from about 500 to over 5,000,000; from about 500 to about 1,000,000; from about 1,000 to about 500,000; from about 2,000 to about 100,000. [0121]In one aspect of the invention, said polymer-BM composition can be utilized, for example, for the rapid detection of target molecules of interest, such as, environmental pollutants, chemical and biological warfare agents and so on, as well as for screening for drug targets and leads, and therapeutic drug and therapeutic impact monitoring.

In another aspect of the invention, said polymer-BM composition can be utilized, for example, for the rapid diagnosis of diseases, such as, cancer, pathological states, as well as for monitoring biomarker changes and protein profiling during life stages, clinical trials and therapeutic treatments.

In another aspect of the invention, said polymer-BM composition can be utilized, for example, for the construction of direct sandwich, indirect sandwich, sequential and competition assays. The assays can be used for either biomarker detection, as well as immunogenicity testing, for example, anti-drug antibody detection.

In yet another aspect of the invention, at least one said polymer can be utilized to carry at least one polypeptide to a solid surface generating virtually no denaturation of the at least one polypeptide. The solid surface can include nitrocellulose, paper, other membranes, glass, metal, a silica-containing device, plastic and the like, and can be presented in a variety of forms, such as flat surfaces, such as, sheets, strips and so on, spheres, such as, particles, beads, and so on, and can be used, for example, for the generation of plate microarrays, bead arrays, microarrays or nanoarrays. The bead micro/nanoarrays either can be constructed through the attachment of multiple molecules, such as, polypeptides of a composition of interest on the same micro/nanoparticle or by having a bead carrying only one species of molecule, such as, a polypeptide of a composition of interest, and mixing beads as desired, wherein each bead carries a specific kind or species of molecule, such as, a polypeptide. In addition to detection, the arrays and assays, such as, bead micro/nanoarrays, also can be utilized for rapid, large-scale, high throughput separation of bioactive materials prior to analysis with protein plate microarrays, 2D gels or mass spectrometers, for example.

As known in the art, assays can be presented in a number of formats, often based on, for example, the separation of reagents on a solid phase and in a liquid phase, formation of molecular bridges and detectable reporter molecules. Hence, binding pairs can play a role in such assays, such as, antibodies, receptors, single-stranded nucleic acids, that may bind by base pairing or by other molecular interaction, such as, forming a triplex nucleic acid or an aptamer, and so on.

As known in the art, a binding pair reagent can be labeled with a detectable reporter, or the detectable reporter can be affixed to another reagent, which can be a member of another binding pair, which indirectly detects the target, for example, to the complex of the target and the binding pair thereof or to the binding pair member bound to the target or to the target, for example. Thus, in some embodiments, two members of mutually exclusive binding pairs each bind the target to form a “sandwich”. Often, one binding pair member is affixed to a solid phase and the other binding pair member may carry the reporter. In such assays, signal level correlates directly with target amount.

In other embodiments, an assay can be configured where the target is tasked with competing for binding to a site with a labeled ligand which also is bound by that site. The target and the ligand can be the same. It follows that in such competition assays, the greater the target concentration, the more likely a target will be bound at the site than a labeled ligand. Thus, in such competition assays, signal correlates inversely with target concentration.

The particular configuration and the particular reagents used in an assay are a design choice based on methods known in the art, reagents known in the art and taught herein and the binding reactions that provide the mechanism of the assay.

As a binding pair comprises receptors, lectins, nucleic acids and so on, the reactants of an assay can comprise any such binding pair as a design choice. Hence a binding pair can comprise a receptor and a hormone, a lectin and a molecule comprising the cognate carbohydrate, complementary nucleic acids, nucleic acids that bind in a fashion similar to that of an antibody, such as, an aptamer, and so on. A combination of different types of binding pairs can be employed in an assay. For example, a nucleic acid may bind a target complement thereof. The detecting nucleic acid comprises a nucleoprotein bound thereto. A solid phase may comprise an antibody which specifically binds the nucleoprotein, and so on.

The composites taught herein can be employed in such assays, for example, the initiator can comprise a reporter molecule and B may be a member of a binding pair that directly or indirectly binds the target.

The polymer compositions of interest can be used as drug delivery devices, which can provide bolus delivery, sustained release, delayed release, timed release, enteric coating and various other pharmacological formulations of desired characteristics. Such composite molecules may also be utilized as sensing components in various sensor platforms including, but not limited to, optical, electrical and piezoelectric devices, as well as in microfluidics, and in microelectromechanical systems (MEMS) and nanoelectromechanical systems (NEMS).

The invention now will be exemplified in the following non-limiting examples.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Materials

All chemicals were available commercially, such as, methylacrylate (MA), ethylenediamine (EDA), piperazine, cyclen, tris(2-aminoethyl) amine (tren), oligomeric ethyleneimines, methyloxazoline, dimethylformamide (DMF), ethyloxazoline, morpholine, N,N′-carbonyldiimidazole (CDI), 4-(aminomethyl) piperidine and dithiothreitol (DTT) were purchased from Sigma-Aldrich. Symmetrically and asymmetrically branched polymers were prepared according to procedures provided in U.S. Pat. Nos. 4,631,337; 5,773,527; 5,631,329; 5,919,442; and 7,754,500. All of the antibodies were purchased from Sigma-Aldrich, Biodesign or Fitzgerald.

### Polymer and Polymer Composite Size Measurement

The size of various polymers and polymer-BM compositions was measured by a size exclusion chromatography and a dynamic light scattering method using a Malvern Zetasizer Nano-ZS Zen3600 particle size analyzer.

### Activity Testing

Metabolism in viable cells produces “reducing equivalents”, such as, NADH or NADPH. Such reducing compounds pass electrons to an intermediate electron transfer reagent that can reduce the tetrazolium product, MTS (Promega), into an aqueous, soluble formazan product, which is colored. At death, cells rapidly lose the ability to reduce tetrazolium products. The production of the colored formazan product, therefore, is proportional to the number of viable cells in culture. The CellTiter 96.RTM. AQueous system (Promega) is an MTS assay for determining the number of viable cells in culture. A single reagent added directly to the culture wells at the recommended ratio of 20 μl reagent to 100 μl of culture medium was used. Cells were incubated 1-4 hours at 37° C. and then absorbance was measured at 490 nm.

Thus, the cytotoxicity of various polymer-drug compositions of interest, along with commercially available drugs or their derivatives, was tested on different cancer cell lines (from ATCC) including, lung cancer cell lines, H460 and A549, and breast cancer lines, MDA-MB-231 and MCF-7, at concentrations ranging from 0.5 mg/mL to 2.5 ng/mL.

Drug-containing nanoparticles of interest comprising a branched polymer, such as, a POX polymer of interest and a PAA, were at least the same or were more potent at killing cancer cells, particularly at low drug concentrations, than PAA alone.

Synthesis of Alkyl-Modified Random (ran) Asymmetrically Branched (AB) Poly(2-ethyloxazoline) (PEOX) with Primary Amine Chain End Group

The synthesis of CH₃-(CH₂)-PEOX-ABP100 (ABP100 is an arbitrary designation to denote the ratio of asymmetrically branched polymer (ABP) monomer to initiator in the initial reaction mixture. Hence, in the above, there is a 100:1 ratio of EOX to initiator. The following synthesis scheme is provided as a general procedure for the preparation of other compositions of interest.

A mixture of 1-bromooctadecane (CH₃(CH₂)₁₇Br) (2.52 g) in 500 ml of toluene was azeotroped to remove water. 2-Ethyloxazoline (100 g) was added dropwise through an addition funnel and the mixture was allowed to reflux between 24 and 48 hours. On completion of polymerization, 12.12 g of EDA were added to the reactive polymer solution to introduce an amine functional group. The molar ratio of polyoxazoline chain end to EDA was about 1 to 20.

N-tert-butyloxycarbonylpiperazine (N-Boc-piperazine) or water (e.g., with 1N Na₂CO₃) can be added to terminate the reaction. Morpholine also can be added to the polymer solution to terminate the reaction. The crude product was redissolved in methanol and then precipitated from a large excess of diethyl ether. The bottom layer was redissolved in methanol and dried by rotary evaporation and vacuum to yield an asymmetrically random branched PEOX polymer as a white solid (101 g). Other asymmetrically randomly branched polymers, such as, C₆-PEOX (using, for example, 1-bromohexane) ABP20, 50, 100, 200, 300 or 500, C₁₈-PEOX (using, for example, 1-bromooctadecane) ABP20, 50, 100, 200, 300 or 500, C₂₂-PEOX (using, for example, 1-bromodocosane) ABP20, 50, 100, 200, 300 or 500, etc., were prepared in a similar manner. All the products were analyzed by size exclusion chromatography (SEC) and nuclear magnetic resonance (NMR).

Synthesis of Linear Poly (2-ethyloxazoline) (PEOX) with Primary Amine Chain End Group

The synthesis of linear HPEOX100 (H is hydrogen) is provided as a general procedure for the preparation of linear POX with a primary amine chain end group. A mixture of p-toluenesulfonic acid monohydrate (FW=190.22; 1.92 g) in 500 ml of toluene was azeotroped to remove water. 2-Ethyloxazoline (100 g) was added dropwise through an addition funnel and the mixture was allowed to reflux about 6 hours. On completion of the polymerization, 12.12 g of EDA were added to the reactive polymer solution to introduce an amine functional group. The molar ratio of polyoxazoline chain end to EDA was about 1 to 20.

N-tert-butyloxycarbonylpiperazine (N-Boc-piperazine) or water (e.g., with 1N Na₂CO₃) can be added to terminate the reaction. Morpholine also can be added to the reactive polymer solution to terminate the reaction. The crude product was redissolved in methanol and then precipitated from a large excess of diethyl ether. The bottom layer was redissolved in methanol and dried by rotary evaporation and vacuum to give an asymmetrically random branched PEOX polymer as a white solid (101 g). Other POX polymers, initiated by toluenesulfonic acid, such as, linear HPEOX 20, 50, 100, 200, 300 or 500, as well as those initiated by methyl tosylate, such as, linear CH₃-PEOX 20, 50, 200, 300 or 500 etc., were prepared in a similar manner. All the products were analyzed by SEC and NMR.

### Synthesis of PEOX-Gemcitabine Composition

An EDA-terminated C₁₈PEOX100 polymer (see above) (1 gram) was dissolved in 10 mL of methanol. Glycidol (15 mg) was added to the solution and mixed. The solution then was incubated at 40° C. for 2 hours. The resulting polymer was extensively dialyzed against water and then dried using a rotary evaporator. The dry polymer (0.93 g) was dissolved in 6 mL of anhydrous DMF.

The polymer solution (2 mL) was mixed with 97 mg of N,N′-carbonyldiimidazole (CDI) and incubated at 37° C. for 2 hours. The resultant product was precipitated by mixing the reaction mixture with 220 mL of diethyl ether at 4° C. for 16 hours. After removing solvent, gemcitabine (41 mg) was added with 9 mL of water. Sodium carbonate (1 M, 1 mL) was added and the resulting solution was incubated at 4° C. for 22 hours. The resulting polymer-gemcitabine composition was purified by dialysis against water. Water then was removed with a rotary evaporator. The composite molecule was redissolved in 5 mL of water and then frozen at −70° C.

### Testing H460 Lung Cell Survival Rate Following PEOX-Gemcitabine Exposure

H460 cells were suspended at 2000 cells/200 μL in RPMI-1640 medium supplemented with 10% fetal bovine serum, 100 IU/mL penicillin and 100 μg/mL streptomycin. The cells were seeded in the wells of a 96-well microplate. The microplate was incubated for 72 hours at 37° C. with 5% CO₂ in air. Thereafter, the culture medium was replaced with 100 μL of fresh cell culture medium containing 0-10,000 ng/ml of gemcitabine or PEOX-gemcitabine composite with an equivalent amount of gemcitabine. All tests were performed in triplicate. Cells were incubated for 72 hours at 37° C. in 5% CO₂ in air. CellTiter 96.RTM. AQueous One Solution Cell Proliferation Assay (20 μL) from Promega mixed with 100 μL of fresh cell culture medium were added to each well and the plate was incubated for 1 hour at 37° C. The absorbance at 490 nm then was measured using a BioTek EPOCH ELISA plate reader.

### Synthesis of PEOX-Camptothecin Composition

Camptothecin (3.48 mg) was added to 1 mL of methylene chloride. CDI (1.62 mg) was added to the mixture which then was stirred at room temperature for 1 hour. EDA-terminated C₁₈PEOX100polymer (see above) (150 mg) was added and incubated at room temperature for 16 hours. The solvent was evaporated to dryness on a rotary evaporator. The resultant solid was redissolved in 3 mL of water, mixed, then filtered through a 0.8 μm syringe filter. The filtrate was frozen at −70° C. for at least 2 hours in a lyophilization vial, then lyophilized overnight (about 16 hours). The ready-to-use white powder was stored at −70° C.

### Testing MCF-7 Breast Cancer and H460 Lung Cell Survival Rate Following PEOX-Camptothecin Exposure

MCF-7 cells were suspended at a density of 6000 cells/200 μL of Eagle's Minimum Essential Medium supplemented with 0.01 mg/mL bovine insulin, 10% fetal bovine serum, 100 IU/mL penicillin and 100 μg/mL streptomycin.

H460 cells were suspended at 2000 cells/200 μL of RPMI-1640 medium supplemented with 10% fetal bovine serum, 100 IU/mL penicillin and 100 μg/mL streptomycin.

The cells were seeded in the wells of a 96-well microplate. The microplate was incubated for 72 hours at 37° C. in 5% CO₂ in air. Thereafter, the culture medium was replaced with 100 μL of fresh cell culture medium containing 0-20 μM of camptothecin or PEOX-camptothecin composite, with an equivalent amount of camptothecin. All tests were performed in triplicate. Cells were incubated for 72 hours at 37° C. in 5% CO₂ in air. CellTiter 96.RTM. AQueous One Solution Cell Proliferation Assay (20 μL) was mixed with 100 μL of fresh cell culture medium and added to each well and the plate was incubated for 1 hour at 37° C. The absorbance at 490 nm then was measured using a BioTek EPOCH ELISA plate reader.

### LC-SPDP-PEOX-NH₂

To EDA-terminated HPEOX100 polymer, see above, (MW 20000, 20 mg or 1×10⁻⁶ mol) in 200 uL of phosphate buffer (20 mM phosphate and 0.1 M NaCl, pH 7.5) were added 10×10⁻⁶ mol of sulfo-LC-SPDP (Thermo, Rockford, III.) in 400 μL of water. The mixture was vortexed and incubated at 30° C. for 30 minutes. The LC-SPDP-PEOX-NH₂ was purified by gel filtration chromatography and equilibrated with buffer A (0.1 M phosphate, 0.1 M NaCl and 5 mM EDTA, pH 6.8). The product was concentrated further to yield 500 μL of solution with a concentration of approximately 1.8 nmol/μL.

### Preparation of Carboxyl End-Functionalized PEOX

To EDA-terminated H-PEOX100 polymer, see above, (MW 20000, 20 mg or 1×10⁻⁶ mol) in 200 uL of methanol were added 1×10⁻⁵ mol of succinic anhydride (Sigma). The mixture was vortexed and incubated at 40° C. for 120 minutes. The H-PEOX-COOH was purified by dialysis. The water was evaporated to dryness on a rotary evaporator.

### Preparation of NHS End-Functionalized PEOX

To the carboxyl end-functionalized H-PEOX100 polymer prepared as taught above (MW 20000, 20 mg or 1×10⁻⁶ mol) in 200 uL of methylene chloride were added 2×10⁻⁶ mol of N,N′-dicyclohexylcarbodiimide (Sigma) and 2×10⁻⁶ mol of N-hydroxysuccinimide (Sigma), and the mixture was incubated at room temperature for 6 hours. After filtration, the reaction mixture was precipitated into diethyl ether at 4° C. for 16 hours. The solvent then was removed.

### Preparation of PEOX/C-reactive Protein Composite

To C-reactive protein (Fitzgerald, Acton, Mass., 2×10⁻⁷ mol) in 5 mL of phosphate buffer (100 mM phosphate and 0.1 M NaCl, pH 7.2) were added 1×10⁻⁶ mol of NHS end-functionalized PEOX polymer prepared as provided above. The reaction was incubated at room temperature for 1 hour. The composite was fractionated on a CM cellulose column (5 ml) with a sodium chloride step gradient in 20 mM phosphate buffer at pH 6. The composite was eluted with a sodium chloride gradient and characterized by ionic exchange chromatography, UV spectroscopy and polyacrylamide gel electrophoresis.

Thiolated PEOX-NH₂ from LC-SPDP-PEOX-NH₂

The LC-SPDP-HPEOX100-NH₂ as described above (50 nmol in 65 ml of buffer A) was mixed with 100 μL of DTT (50 mM in buffer A) and the mixture was allowed to incubate at room temperature for 15 minutes. Excess DTT and byproducts were then removed by gel filtration with buffer A. The product was concentrated in a 10 K Centricon Concentrator to yield 450 μL of thiolated PEOX-NH₂ that was used for joining with maleimide-R-activated antibody made as described below.

Maleimide R (MAL-R)-activated Antibody

To antibody in PBS (310 μL, 5.1 mg or 34 nmol) were added 20.4 μL of a MAL-R-NHS (N-hydroxysuccinimide) solution (10 mM in water) (succinimidyl-[(N-maleimidopropionamido)-tetracosaethyleneglycol] ester purchased from ThermoFisher). The mixture was vortexed and incubated at 30° C. for 15 minutes. The product was purified by gel filtration with buffer A. The maleimide-R-activated antibody was used for joining with thiolated compounds, such as, thiolated HPEOX100-NH₂.

### HPEOX-Antibody Composition

To the thiolated HPEOX100-NH₂ prepared as taught above (350 μL or 35 nmol) was added MAL-R-activated antibody (4.8 mL or 34 nmol). The reaction mixture was concentrated to approximately 800 μL and allowed to incubate overnight at 4° C. or at room temperature for about 1 hr. On completion, the reaction was quenched with 100 μL of ethyl maleimide (50 mmolar solution) and the composition then was fractionated on a CM cellulose column (5 ml) with a sodium chloride step gradient in 20 mM phosphate buffer at pH 6. The composition was eluted with a sodium chloride gradient and characterized by ionic exchange chromatography, UV spectroscopy and polyacrylamide gel electrophoresis.

### Reduction of Antibody

To antibody, 2.1 mg or 14 nmol in 160 μL of buffer B (containing 0.1 M sodium phosphate, 5 mM EDTA and 0.1 M NaCl, pH 6.0) were added 40 μL of DTT (50 mM in buffer B). The solution was allowed to stand at room temperature for 30 min. The product was purified by gel filtration over a Sephadex G-25 column equilibrated with buffer B. The reduced antibody was concentrated to 220 μL and was used for joining with other molecules.

### MAL-R-HPEOX

To EDA-terminated HPEOX in 400 μL (400×10⁻⁹ mols) at pH 7.4 were added 400 μL of MAL-PEG₂₄-NHS (Quanta BioDesign, Powell, Ohio) (10 mM in water). That was mixed and incubated at 30° C. for 15 min. On termination, the product was purified on a Sephadex G-25 column equilibrated with buffer B. The MAL-R-PEOX was collected and stored in aliquots in the same buffer at 40° C.

### HPEOX-Antibody Composition

To the reduced antibody described above (14 nmols in 220 μL) was added the MAL-R-HPEOX (154 μL, 16.6 nmols) with stirring. To that were added 12.5 μL of sodium carbonate (1.0 M solution) to bring the pH to about 6.8. The reaction was continued for 1 hr at room temperature. On completion, the reaction was quenched with 100 μL of cysteamine (0.4 mM solution) and the composition then was fractionated on a CM cellulose column (5 ml) with a sodium chloride step gradient in 20 mM phosphate buffer at pH 6. The composition was eluted with a sodium chloride gradient and characterized by ionic exchange chromatography, UV spectroscopy and polyacrylamide gel electrophoresis.

### Synthesis of Random Asymmetrically Branched PEOX-PAMAM-1 Copolymer

Random asymmetrically branched C₁₈-PEOX-100-NH₂ (MW=30,000), methyl acrylate (MA, FW=86.09), ethylenediamine (EDA, FW=60.10), monoethanolamine (MEA, FW=61.08) and methanol were used.

To a round bottom flask were added 10.0 g C₁₈-PEOX-100-NH₂ and 100 ml methanol (solution A). To a separate round bottom flask were added 86 mg methylacrylate (MA) and 1 ml methanol (solution B). Solution A was then slowly dropped into solution B while stirring at room temperature. The resulting solution was allowed to react at 40° C. for 2 hours. On completion of the reaction, the solvent and unreacted monomer, MA, were removed by rotary evaporation, and the product, MA functionalized C₁₈-PEOX-100-NH₂, was then redissolved in 100 ml of methanol.

To a round bottom flask were added 5 g EDA and 50 ml of methanol, followed by a slow addition of MA-functionalized C₁₈-PEOx-100-NH₂ at 0° C. (1 g MA functionalized C₁₈-PEOX-100-NH₂ dissolved in 10 ml methanol). The solution was then allowed to react at 4° C. for 48 hours. The solvent and the excess EDA were removed by rotary evaporation. The crude product was then precipitated from an ethyl ether solution, and further purified by dialysis to give about 10.076 g random asymmetrically branched PEOX-PAMAM-1.0 copolymer, the theoretical molecular weight is about 30,228. The product was characterized by ¹H and ¹³C nuclear magnetic resonance (NMR) and size exclusion chromatography (SEC).

Other polymers, such as, random asymmetrically branched PEOX-PAMAM-2 (with 2 PAMAM layers and NH₂ as surface groups), ran-PEOX-PAMAM-3 (with 3 PAMAM layers and NH₂ as surface groups), etc, were prepared by repeating the above synthetic steps, for example, the addition of MA, followed by the reaction with a large excess of EDA. Alternatively, ran-PEOX-PAMAM-2 (OH) (with 2 PAMAM layers and OH as surface groups), and ran-PEOX-PAMAM-3 (OH) (with 3 PAMAM layers and OH as surface groups) and so on can be produced by repeating the above synthetic steps, for example, the addition of MA, followed by reaction with a large excess of MEA.

Additionally, ran-PEOX-PAMAM-2 (NH₂/OH) (with 2 PAMAM layers and a mixture of NH₂ and OH as surface groups), and ran-PEOX-PAMAM-3 (NH₂/OH) (with 3 PAMAM layers and NH₂/OH mix as surface groups) can be prepared by repeating the above synthetic steps, for example, the addition of MA, followed by reaction with a mixture of a large excess of EDA/MEA.

What is claimed is:

1. A functionalized polymer comprising a polyoxazoline polymer P reacted with at least one initiator I and a first linker L, wherein said L is further reacted with two or more second linker M, and wherein,
said initiator I comprises a first functional group that is reacted with P and a second functional group that is reactive with a first biologically active molecule B1,
said first linker L comprises three or more active hydrogens in amine groups selected from an NH₃, two or more —NH₂ groups, three or more imino (—NH—) groups, or a combination of —NH₂ and —NH—groups, wherein said first linker L is reacted with said P with one of said active hydrogens;
said M comprises a carbodiimide (CDI), a CDI-functionalized molecule, glycidol (G), succinic anhydride (SA), acrylic ester, acrylamide or a heterofunctional molecule, said M comprises at least one first linker group reacted with one of the active hydrogens of said L and at least one second linker group reactive with a second biologically active molecule B2; and
said functionalized polymer comprises a formula (1):
Iₙ—P—L—(M)_{y}
wherein, n and y each is an integer,
n≥1, and
y≥2.

2. The functionalized polymer of any one of claims 1, further comprising at least one said first biologically active molecule B1 reacted with said initiator I, at least one said second biologically active molecule B2 reacted with at least one of said M, or a combination thereof.

3. The functionalized polymer of claim 2 comprising a formula (2):
(B1)ₓ—Iₙ—P—L—(M—(B2)_{z})_{y}
a formula (3):
((B1)ₓ—I)ₙ—P—L—(M—(B2)_{z})_{y}
a formula (4):
(B1)ₓ—Iₙ—P—L—(M)_{y}—(B2)_{z}
or a formula (5):
((B1)ₓ—I)ₙ—P—L—(M)_{y}—(B2)_{z}
n, x, y and z each is an integer and
x≥0,
n≥1,
y≥2,
z≥0,
with a proviso that one of x or z is not 0.

4. The functionalized polymer of claim 2, wherein said B1 or B2 each independently comprises a member of a binding pair (BP), a bioactive material (BM), a biologically active molecule (BAM), or a pharmaceutically active agent (PAA).

5. The functionalized polymer of claim 4, wherein said B1 or B2 each independently comprises a small molecule drug, a large molecule drug, a chemotherapy drug, an immunosuppressant drug, or a combination thereof.

6. The functionalized polymer of claim 5, wherein said large molecule drug comprises a polypeptide, a polynucleotide, an antibody, a monoclonal antibody (mAb) or a combination thereof.

7. The functionalized polymer of claim 5, wherein said small molecule drug comprises a taxane, a paclitaxel, a gemcitabine, a camptothecin, anthracyclines, doxorubicin, epirubicin, cisplatin, carboplatin, vinorelbine, capecitabine, ixabepilone, eribulin or a combination thereof.

8. The functionalized polymer of claim 4, wherein said B1 is a monoclonal antibody selected from abciximab, adalimumab, alefacept, alemtuzumab, alirocumab, apatinib, arcitumomab, atezolizumab, avelumab, basiliximab, belimumab, besilesomab, bevacizumab, bezlotoxumab, brentuximab, brodalumab, canakinumab, capromab, catumaxomab, certolizumab, cetuximab, daclizumab, daratumumab, denosumab, dinutuximab, dupilumab, durvalumab, eculizumab, efalizumab, efalizumab, elotuzumab, evolocumab, fanolesomab, gemtuzumab, golimumab, herceptin adc, ibritumomab, ibritumomab tiuxetan, idarucizumab, imiciromab, inflectra, infliximab, ipilimumab, ixekizumab, lifirafenib, mepolizumab, muromonab-cd3, natalizumab, necitumumab, nivolumab, nofetumomab, obiltoxaximab, obinutuzumab, ocrelizumab, ofatumumab, olaratumab, omalizumab, osimertinib, palivizumab, pamiparib, panitumumab, pembrolizumab, pertuzumab, pyrotinib, ramucirumab, ranibizumab, raxibacumab, reslizumab, rituximab, rituximab, satumomab, secukinumab, shr1210m, shr-a1201, siltuximab, sulesomab, tislelizumab, tocilizumab, tositumomab, trastuzumab, ustekinumab, vedolizumab, votumumab, or zanubrutinib; a cytokine selected from an interleukin, interferon α2a or interferon α; a hormone selected from granulocyte colony stimulating factor (GCSF), insulin, glucagon, glucagon like peptide-1, erythropoietin or follicle stimulating hormone; a ligand of cell surface receptors; a lectin; nucleic acids selected from an siRNA, a ribozyme, antisense nucleic acids, naked nucleic acids, a CpG oligonucleotide (ODN); a virus; or a virus-like particle; and said B2 comprises a taxane, a paclitaxel, a gemcitabine, a camptothecin, anthracyclines, doxorubicin, epirubicin, cisplatin, carboplatin, vinorelbine, capecitabine, ixabepilone or eribulin.

9. The functionalized polymer of claim 2, wherein said B1 or B2 each comprises a targeting moiety.

10. The functionalized polymer of claim 9, wherein said targeting moiety comprises an antibody or an antigen-binding portion thereof.

11. The functionalized polymer of claim 2, wherein said B2 is linked to M through a cleavable linkage.

12. The functionalized polymer of claim 2, wherein said B2 is linked to M through an anhydride bond or a peptide bond.

13. The functionalized polymer of claim 1, wherein said first functional group comprises sulfonic acid, organic acid, alkyl halide, allyl halide, benzyl halide, tosylate, p-alkyl toxylate or p-toluenesulfonic acid.

14. The functionalized polymer of claim 1, wherein said second functional group comprises an ester group, an amide group, a maleimide group, a ketone group, an aldehyde group, a —SH group, an —S—S— containing group, a thiolated group, a benzyl group, a protected amino group, a carboxybenzyl group, a p-methoxybenzyl carbonyl group, a tert-butyloxycarbonyl group, a 9-fluorenylmethyloxycarbonyl (FMOC) group, a silicon containing group, or a combination thereof.

15. The functionalized polymer of claim 1, where said initiator comprises ethyl bromoacetate, methyl bromoacetate, bromoacetone, tert-butyl bromoacetate, propyl bromoacetate, benzyl bromoacetate, a silicon-containing compound and a protected amine or imine.

16. The functionalized polymer of claim 1, wherein said P comprises a poly(unsubstituted oxazoline), a poly(substituted oxazoline), or a combination thereof, wherein said poly(substituted oxazoline) comprises a poly(2 methyloxazoline), a poly(2-ethyloxazoline), a poly(2-propyloxazoline), a poly(2 butyloxazoline) or a combination thereof.

17. The functionalized polymer of claim 1, wherein said P is a linear polymer, a dendritic polymer, a randomly branched polymer, or a combination thereof.

18. The functionalized polymer of claim 1, wherein said L comprises a reacted NH₃, ethylenediamine, tris(2 aminoethyl)amine, 4 (aminomethyl)piperidine, 1,3-diaminopropane, 2,2′ (ethylenedioxy)bis(ethylamine), diethylenetriamine, 1,4,7,10-tetraazacyclododecane, hexamethylenediamine, triethylenetetramine, or 1,8-diaminooctane.

19. The functionalized polymer of claim 1, wherein said L comprises a NH₃ having 3 active hydrogens, wherein one of said active hydrogens is reacted with said P and the remaining active hydrogens are reacted to 2 or more M.

20. The functionalized polymer of claim 1, wherein said L comprises at least two —NH₂ groups having 4 or more active hydrogens, wherein one of said active hydrogens is reacted with said P and the remaining active hydrogens are reacted to 2 or more M.

21. The functionalized polymer of claim 1, wherein said L comprises at least one —NH₂ group and at least one —NH group having 3 or more active hydrogens, wherein one of said active hydrogens is reacted with said P and the remaining active hydrogens are reacted to 2 or more M.

22. The functionalized polymer of claim 1, wherein said L comprises at least three —NH groups having 3 or more active hydrogens, wherein one of said active hydrogens is reacted with said P and the remaining active hydrogens are reacted to 2 or more M.
